(19) United States
(12) Patent Application Publication
(10) Pub. No.: US 2025/0146051 A1
(43) Pub. Date: May 8, 2025

(54) METHODS FOR IDENTIFYING RISK OF AUTISM

(71) Applicant: The Johns Hopkins University, Baltimore, MD (US)

(72) Inventors: Andrew Paul Feinberg, Baltimore, MD (US); Heather Volk, Baltimore, MD (US); Jason Ira Feinberg, Baltimore, MD (US); Rose Schrott, Baltimore, MD (US)

(21) Appl. No.: 18/838,389

(22) PCT Filed: Feb. 27, 2023
(86) PCT No.: PCT/US2023/013998

§ 371 Date: Aug. 14, 2024

Related U.S. Application Data
(60) Provisional application No. 63/315,000, filed on Feb. 28, 2022.

(51) Int. Cl. C12Q 1/6809; C12Q 1/6883

(52) U.S. Cl. CPC C12Q 1/6809; C12Q 1/6883; C12Q 2600/154

(57) ABSTRACT

The invention provides herein epigenetics changes identified in differentially methylated regions (DMRs) of DNA of a father that are associated with the risk for an offspring having autism spectrum disorder (ASD). The invention further provides methods of determining a risk of having an offspring with ASD, methods of diagnosing ASD in a subject, and methods of determining an association between exposure to an environmental factor in a subject and an increased risk of having an offspring with ASD. The invention also provides a kit for determining whether a subject has or is at risk of having or inheriting a risk of having ASD.

### CROSS-REFERENCE TO RELATED APPLICATIONS

This application claims benefit of priority under 35 U.S.C. § 119 (e) of U.S. Provisional Application No. 63/315,000, filed Feb. 28, 2022. The disclosure of the prior application is considered part of and are herein incorporated by reference in the disclosure of this application in its entirety.

### STATEMENT REGARDING FEDERALLY SPONSORED RESEARCH OR DEVELOPMENT

This invention was made with government support under grant ES017646 awarded by the National Institutes of Health. The government has certain rights in the invention.

### BACKGROUND OF THE INVENTION

### Field of the Invention

The present invention relates generally to epigenetics and more specifically to determining the risk for an offspring to have autism spectrum disorder (ASD).

### Background Information

Autism etiology is complex and heritability is not explained by genetics alone. Understanding paternal gametic epigenetic contributions to autism could help fill this knowledge gap. While evidence to support paternal contributions to offspring autism spectrum disorder (ASD) is mounting, ASD etiology is complex-genetic influences are heterogenous, and the precise contributions of environmental factors are not well known. Genetic variation, family history of ASD and other psychopathology, and trait-based metrics can contribute to ASD occurrence and ASD-related trait severity. Both genetic variations and quantitative autistic traits (QATs) contribute to the known heritability of ASD and autistic characteristics, respectively, though neither is solely responsible for the intergenerational transmission of this multifaceted disorder.

Gametic epigenetic modifications can reflect both genetic and environmental variation, have been implicated in ASD, and might provide an additional pathway for ASD heritability. Specifically, DNA methylation, characterized by the presence of a methyl group at a cytosine base when cytosine is followed by guanine in the nucleotide sequence (referred to as a CpG site), is the most well-characterized type of epigenetic modification, and studies of methylation have provided the greatest support to date when addressing how paternal epigenetic changes might be associated with offspring ASD risk.

The establishment and maintenance of germline DNA methylation is essential to spermatogenesis. Methylation remodeling occurs throughout multiple stages of sperm maturation, highlighting both the need for methylation to be faithfully sustained during spermatogenesis, as well as the potential vulnerability of the epigenome to the exogenous environment as it is in a labile state to allow the requisite methylation changes to occur. Indeed, the sperm epigenome changes in response to environmental exposures and periods of stress. Work in animal models, as well as epidemiologic studies, have begun to demonstrate that epigenetic changes in sperm result from a variety of exposures, some of which are further associated with epigenetic changes and adverse developmental outcomes in offspring. Thus, changes to the sperm epigenome are perhaps one mode whereby paternal germline factors can influence offspring neurodevelopment and ASD.

It has been examined how changes to the sperm methylome might be related to autism, and ASD-related quantitative traits in children. Specifically, it was previously reported that paternal sperm DNA methylation changes were associated with ASD-related outcomes in 12-month-olds enrolled in the Early Autism Risk Longitudinal Investigation (EARLI) study. EARLI is focused on the siblings of children who have already been diagnosed with autism given that disorders like ASD have higher rates of familial aggregation, particularly among siblings. Climbing rates of ASD in the United States (US) accentuate the need to better understand paternal contributions to ASD etiology.

### SUMMARY OF THE INVENTION

The present invention is based on the seminal discovery that epigenetics changes in differentially methylated regions (DMRs) of DNA of a father are associated with the risk for an offspring of having autism spectrum disorder (ASD).

The present invention details the role of the paternal germline epigenome in contributing to offspring autistic traits in EARLI. The Social Responsiveness Scale (SRS)—a questionnaire that is designed to assess social functioning and social abilities—was used as a measure of QATs in EARLI fathers and children. We first asked whether there was a relationship between paternal and offspring SRS scores. DNA methylation was measured in paternal sperm to examine whether genome-scale methylation was associated with 36-month child SRS scores. We then examined whether sperm DNA methylation was associated with SRS scores in fathers themselves. Once significant DMRs were identified, we determined whether there were commonalities between DMRs associated with paternal and child SRS scores. Finally, we compared child SRS-associated DMRs to our previously published work in these same 12-month-old EARLI participants, as well as to an independent methylation dataset consisting of post-mortem human brain tissue from individuals with ASD and controls.

In one embodiment, the invention provides a method of determining a risk of having an offspring with autism spectrum disorder (ASD) including: a) measuring DNA methylation status at differentially methylated regions (DMRs) in DNA from a semen sample from a paternal subject; and b) determining a risk score based on DMRs methylation status, thereby determining a risk of having an offspring with ASD.

In one aspect, a difference in the DNA methylation status at one or more DMRs as compared to a control DNA methylation status is indicative of a higher likelihood of having an offspring with ASD than a likelihood of having an offspring with ASD as measured in the control DNA. In some aspects, the control DNA methylation status is a DNA methylation status at the one or more DMRs measured in a subject that is not at risk of having an offspring with ASD. In other aspects, the subject is a prospective parent. In some aspects, the subject has a risk factor for having an offspring with ASD. In other aspects, determining a risk of having an offspring with ASD includes predicting a risk of having an offspring with features of autism as measured by a social responsiveness scale (SRS) score.

In another aspect, the invention provides a method of diagnosing autism spectrum disorder (ASD) in a subject including: a) measuring a DNA methylation status at one or more differentially methylated regions (DMRs) in DNA sample from the subject; and b) determining a risk score based on DMRs methylation status, thereby diagnosing ASD in the subject.

In one aspect, a difference in the DNA methylation status at one or more DMRs as compared to a control DNA methylation status is indicative of a higher likelihood of having ASD than a likelihood of having ASD as measured in the control DNA. In another aspect, the DMRs are in genes selected from group of genes set forth in Table 6, Table 7 or Table 8. In some aspects, the DMRs include at least 1, at least 2, at least 3, at least 4, at least 5, at least 6, at least 7, at least 8, at least 9, at least 10, at least 11, at least 12, at least 13, or 14 or more genes from Table 6. In another aspect, the DMRs include at least 1, at least 2, at least 3, at least 4, at least 5, at least 6, at least 7, at least 8, at least 9, at least 10, at least 11, at least 12, at least 13, or 14 genes from Table 8. In some aspects, the DMRs include 3 to 15 DMRs. In some aspects, the genes are ASD-associated genes. In one aspect, a difference in the DNA methylation status includes hypomethylation, hypermethylation or a combination thereof. In various aspects, the subject is human. In some aspects, measuring DNA methylation status is by methylation specific PCR, bisulfite sequencing, capture bisulfite sequencing, whole genome bisulfite sequencing, pyrosequencing, single-strand conformation polymorphism (SSCP) analysis, restriction analysis, microarray technology, including bead microarray technology, or proteomics. In other aspects, the DNA methylation status at the one or more DMRs is associated with an SRS score in the subject. In other aspects the DNA methylation status at the one or more DMRs is associated with an SRS score in the offspring.

In an additional embodiment, the invention provides a method of determining an association between exposure to an environmental factor in a subject and an increased risk of having an offspring with autism spectrum disorder (ASD) including: a) measuring a first DNA methylation status at differentially methylated regions (DMRs) in DNA from a first semen sample from the subject prior to exposure to the environmental factor; b) measuring a second DNA methylation status at DMRs in DNA from a second semen sample from the subject after to exposure to the environmental factor; and c) comparing the first and the second methylation status at the DMRs; thereby determining an association between exposure to an environmental factor and an increased risk of having an offspring with ASD.

In one aspect, a change in the methylation status at DMRs between the first DNA methylation status and the second DNA methylation status in indicative of an association between the environmental factor and an increased risk of having an offspring with autism ASD.

In various aspects, the DNA is from sperm in the semen sample.

In one embodiment, the invention provides a kit for determining whether a subject has or is at risk of having or inheriting a risk of having autism spectrum disorder (ASD), including: a) a reagent for determining a DNA methylation status at one or more differentially methylated regions (DMRs) in a DNA sample from the subject; and b) instructions for use of the reagent. In one aspects, the one or more DMRs are selected from genes set forth in Table 6, Table 7, and Table 8.

### BRIEF DESCRIPTION OF THE DRAWINGS

FIGS. 1A-1B. FIG. 1A is a graph showing CHARM versus 450K cross-platform validation in child SRS DMRs. FIG. 1B is a graph showing CHARM versus 450K cross-platform validation in dad SRS DMRs. X-axis shows the CHARM value returned by bump hunting for 36-month child/paternal SRS DMRs.-axis shows the mean 450k regression coefficients resulting from modeling SRS scores on DNAm, assessed for each 450k probe overlapping within 500 bp of a CHARM DMR.

FIGS. 2A-2B. FIG. 2A is a volcano plots for 1482 Child SRS DMRs. FIG. 2B is a volcano plots for 1928 Dad SRS DMRs. Y-axis shows the log 10 (FWER P) for each DMR returned by the bump hunter algorithm after 10,000 bootstrap permutations. X-axis is the CHARM DMR value which corresponds to the smoothed effect estimate at each probe. Filled in circles have fwer p<0.05, open circles have fwer p<0.1, and black circles have no nominal significance.

FIGS. 3A-3H illustrate methylation plots for the top four statistical DMRs (P<1.0×10-4) identified using CHARM and 36-month child SRS score. FIG. 3A illustrates methylation plots for WWOX. FIG. 3B illustrates the relationship between WWOX methylation and SRS score. FIG. 3C illustrates methylation plots for A2BP1. FIG. 3D illustrates the relationship between A2BP1 methylation and SRS score. FIG. 3E illustrates methylation plots for SALL3. FIG. 3F illustrates the relationship between SALL3 methylation and SRS score. FIG. 3G illustrates methylation plots for WWOX. FIG. 3H illustrates the relationship between WWOX methylation and SRS score. FIGS. 3A, 3C, 3E, and 3G show individual methylation levels at each probe by genomic position. Dotted vertical lines represent the boundaries of the DMR, and continuous lines represent the average methylation curve for samples grouped by quartiles of SRS scores the scores within each quartile are shown in the legend.

FIGS. 4A-4H illustrate dad SRS DMRs: methylation plots for the top four statistical DMRs (P<1.0×10-4) identified using CHARM and 36-month child SRS score. FIG. 4A illustrates methylation plots for SMYD3. FIG. 4B illustrates the relationship between SMYD3 methylation and SRS score. FIG. 4C illustrates methylation plots for SALL3. FIG. 4D illustrates the relationship between SALL3 methylation and SRS score. FIG. 4E illustrates methylation plots for GUCY2G. FIG. 4F illustrates the relationship between GUCY2G methylation and SRS score. FIG. 4G illustrates methylation plots for TGM3. FIG. 4H illustrates the relationship between TGM3 methylation and SRS score. FIGS. 4A, 4C, 4E, and 4F show individual methylation levels at each probe by genomic position. Dotted vertical lines represent the boundaries of the DMR, and continuous lines represent the average methylation curve for samples grouped by quartiles of SRS scores; the scores within each quartile are shown in the legend. Bottom panel shows location of CpG dinucleotides (as black tick marks) and CpG density by genomic position (black curved line).

FIG. 5 is a schematic representation of the overlaps between child and paternal DMRs related to SRS.

### DETAILED DESCRIPTION OF THE INVENTION

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

As used herein and in the claims, the terms “comprising,” “containing,” and “including” are inclusive, open-ended and do not exclude additional unrecited elements, compositional components or method steps. Accordingly, the terms “comprising” and “including” encompass the comparably more restrictive terms “consisting of” and “consisting essentially of.”

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

The present invention is based on the seminal discovery that paternal epigenetics can be predictive for autism spectrum disorder (ASD) risk in children. While ASD can be responsive to a range of factors, the multifaceted and often incongruous connection between parental and offspring ASD has posed a consistent challenge to ASD risk assessment in children. Exemplifying the tenuousness of paternal characteristics as a predictor for child ASD, the present invention demonstrates that paternal social responsiveness scale (SRS) is not predictive of ASD risk in children (EXAMPLE 2). Nonetheless, it was surprisingly demonstrated herein that epigenetic changes in paternal DNA, including those measurable in gametes, can be predictive for offspring ASD.

As used herein, the terms “autism spectrum disorder” and “autistic spectrum disorder” refer to neurodevelopmental disorders characterized by diminished abilities for socialization and communication. ASD encompasses a myriad of developmental and behavioral impairments, ranging from minor difficulties in communication and social interaction, as in many cases of Asperger's syndrome, to the inability to speak or recognize basic social cues. While ASD can manifest as a range of impairments, subjects with ASD often exhibit increased SRS scores, for example scores of at least 60, 70, 80, 90, or 100. A number of sources provide guidance on ASD diagnosis, including Autism Spectrum Disorders: A Research Review for Practitioners, Ozonoff, et al., eds., 2003, American Psychiatric Pub, and Handbook of Assessment and Diagnosis of Autism Spectrum Disorder, Matson, ed., 2016, Basel: Springer. While ASD development is responsive to a range of environmental, genetic, and, as demonstrated herein, epigenetic factors, early ASD diagnosis can enable treatments and interventions which improve ASD outcomes.

Addressing the need for improved child ASD risk assessment and leveraging the surprising discovery that paternal epigenetics can be predictive for ASD in children, the present invention provides paternal DNA methylation assays for offspring ASD risk assessment. In certain aspects, the present invention provides a method of determining a risk of having an offspring with autism spectrum disorder (ASD) by measuring DNA methylation status at differentially methylated regions (DMRs) in DNA from a semen sample from a paternal subject; and determining a risk score based on DMRs methylation status, thereby determining a risk of having an offspring with ASD.

As used herein, the term “differentially methylated region” (DMR) refers to a region in chromosomal DNA with variable methylation status across multiple samples from a single species or an individual. While many genomic regions exhibit consistent epigenetic profiles, certain regions can vary considerably terms of DNA modification and histonylation. One type of differentially processed region is a differentially methylated region, which may exhibit a difference in methylation density and/or pattern across samples. In some cases, a differentially methylated region exhibits at least about 1-fold, at least 1.01-fold, at least 1.02-fold, at least 1.03-fold, at least 1.04-fold, at least 1.05-fold, at least 1.06-fold, at least 1.07-fold, at least 1.08-fold, at least 1.09-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, at least 5-fold, at least 10-fold, or greater degrees of variation in methylation density across samples. A differentially methylated region may also exhibit at least 5%, at least 10%, at least 20%, at least 30%, at least 40%, at least 50%, at least 60%, at least 70%, at least 80%, or at least 90% difference in methylation pattern between samples. A differentially methylated region can be a gene or a portion of a gene, can encompass a plurality of genes, or can encompass a non-coding region of a genome. In some aspects, a differentially methylated region is selected from among those set forth in Tables 6-8. In some aspects, a differentially methylated region is characterized by variance in methylation density between a control sample and a sample from the subject. In some aspects, a difference in the DNA methylation status includes hypomethylation, hypermethylation or a combination thereof.

In some aspects, determining a risk of having an offspring with ASD includes predicting a risk of having an offspring with features of autism as measured by a social responsiveness scale (SRS) score. The SRS score is designed to assess social functioning and social abilities. Due to the complexity of ASD, SRS score is often used in combination with other tests for ASD diagnosis. Nonetheless, higher SRS scores typically correlate with ASD prevalence and severity, with scores of 60 or higher typically portending a likelihood of ASD, and scores of 100 or higher often indicating moderate or severe ASD. In some cases, determining a risk of having an offspring with ASD includes predicting a risk of having an offspring with an SRS score of at least about 60, at least about 70, at least about 80, at least about 90, at least about 100, at least about 110, or at least about 120.

In certain aspects, DNA methylation status is determined by comparing DNA methylation in DMRs of the paternal semen sample to DNA methylation in one or more control samples. In some aspects, the control DNA methylation status is a DNA methylation status at the one or more DMRs measured in a subject that is not at risk of having an offspring with ASD. As disclosed herein, a difference in the DNA methylation status at one or more DMRs as compared to a control DNA methylation status can also be indicative of a higher likelihood of having an offspring with ASD than a likelihood of having an offspring with ASD as measured in the control DNA. Alternatively or in addition thereto, a difference in the DNA methylation status at one or more DMRs as compared to a control DNA methylation status can also be indicative of a higher likelihood of having an offspring with ASD than a likelihood of having an offspring with ASD as determined for the subject from which the control sample was obtained (e.g., with a questionnaire). In some aspects, the control DNA methylation status is a DNA methylation status at one or more DMRs measured in a plurality of subjects that are not at risk of having an offspring with ASD.

In some aspects, DNA methylation status is measured in at least 3, at least 5, at least 8, at least 12, at least 15, at least 20, at least 25, at least 30, at least 40, at least 50, at least 75, at least 100, at least 150, at least 200, or at least 250 DMRs. In some aspects, DNA methylation status is measured in at most 250, at most 200, at most 150, at most 100, at most 75, at most 50, at most 40, at most 30, at most 25, at most 20, at most 15, at most 12, at most 8, at most 5, or at most 3 DMRs. In some aspects, DNA methylation status is measured in about 3 to 10, in about 3 to 15, in about 5 to 15, in about 5 to 25, in about 8 to 30, in about 12 to 50, in about 20 to 50, in about 20 to 100, in about 50 to 100, or in about 100 to 500 DMRs.

In certain aspects, a control sample is obtained from a parent at risk for having offspring with ASD. In certain aspects, a difference in the DNA methylation status at one or more DMRs as compared to a control DNA methylation status can be indicative of a lower likelihood of having an offspring with ASD than a likelihood of having an offspring with ASD as measured in the control DNA.

In certain aspects, control samples are obtained from a plurality of parents with different risks for having offspring with ASD. For example, the DNA methylation in the control samples may be used to generate a calibration curve to determine ASD risk in offspring of the paternal subject. Accordingly, in certain aspects, the DNA methylation status at one or more DMRs as compared to control DNA methylation statuses can be used to determine the likelihood of having an offspring with ASD.

In some aspects, the DMRs are in genes selected from the group of genes set forth in Table 6, Table 7, or Table 8. In some aspects, the DMRs include a 5′ untranslated region (UTR). In some cases, the DMRs include a 3′ UTR. In some aspects, the DMRs include an intron. In some cases, the DMRs include an exon. In some aspects, the genes include an ASD-associated gene. In some cases, the genes are ASD-associated genes. In some aspects, the genes include a non-ASD-associated genes. In some aspects, the genes are non-ASD-associated genes. In some aspects, the DNA methylation status at the one or more DMRs is associated with an SRS score in the subject. In some aspects, the DNA methylation status at the one or more DMRs is associated with an SRS score in the offspring.

In some cases, the DMRs are in genes selected from the group of genes set forth in Table 6. In some cases, the DMRs include at least 1, at least 2, at least 3, at least 4, at least 5, at least 6, at least 7, at least 8, at least 9, at least 10, at least 11, at least 12, at least 13, or all 14 genes in Table 6. In some cases, the DMRs include a gene associated with synaptic function, neurogenesis, development, or a combination thereof. In some cases, the DMRs include a gene or a 5′ or 3′ untranslated region of a gene associated with WW domain-containing oxidoreductase (e.g., chr16: 79027119-79030054), RNA binding fox-1 homolog 1 (e.g., chr16: 7065694-7068381), spalt like transcription factor 3 (e.g., chr18: 76744751-76746907), Adenosine Deaminase RNA Specific B2 (e.g., chr10: 1894909-1896546), phosphofructokinase (e.g., chr10: 3057233-3058630), zinc finger protein 536 (e.g., chr19: 30745777-30747506), vascular endothelial growth factor C (e.g., chr19: 30745777-30747506), galanin receptor 1 (e.g., chr18: 75687429-75689009), RNA binding fox-1 homolog 1 (e.g., chr16: 6334658-6336272), junctional Adhesion Molecule 3 (e.g., chr11: 133989860-133991280), or a combination thereof. In some aspects, the DMRs include chr16: 79027119-79030054, or a portion thereof. In some aspects, the DMRs include chr16: 7065694-7068381, or a portion thereof. In some aspects, the DMRs include chr18: 76744751-76746907, or a portion thereof. In some aspects, the DMRs include chr16: 78376735-78378445. In some aspects, the DMRs include chr10: 1894909-1896546, or a portion thereof. In some aspects, the DMRs include chr16: 78974715-78976580, or a portion thereof. In some aspects, the DMRs include chr10: 3057233-3058630. In some aspects, the DMRs include chr19: 30745777-30747506, or a portion thereof. In some aspects, the DMRs include chr4: 177804604-177805652. In some aspects, the DMRs include chr18: 75687429-75689009, or a portion thereof. In some aspects, the DMRs include chr16: 6334658-6336272, or a portion thereof. In some aspects, the DMRs include chr11: 133989860-133991280, or a portion thereof. In some aspects, the DMRs include chr14: 99254155-99255193, or a portion thereof. In some aspects, the DMRs include chrX: 103301728-103303214, or a portion thereof. In some aspects, the portion thereof is at least about 25%, at least about 50%, at least about 75%, or at least about 90% of the gene.

In some cases, the DMRs are in genes selected from the group of genes set forth in Table 8. In some cases, the DMRs include at least 1, at least 2, at least 3, at least 4, at least 5, at least 6, at least 7, at least 8, at least 9, at least 10, at least 11, at least 12, at least 13, or all 14 genes in Table 8. In some cases, the DMRs include a gene associated with epigenetic regulation, embryonic development, cellular differentiation, neuronal signaling, or a combination thereof. In some cases, the DMRs include a gene or a 5′ or 3′ untranslated region of a gene associated with SET and MYND domain-containing protein 3 (e.g., chr1: 246058026-246059550), spalt like transcription factor 3 (e.g., chr18: 76744751-76746907), transglutaminase 3 (e.g., chr20: 2216856-2218239), WW Domain Containing Oxidoreductase (e.g., chr16: 79027537-79029698), neuronal differentiation 2 (e.g., chr17: 37756945-37758433), zinc finger protein 32 (e.g., chr10: 44173499-44175059), Ankyrin Repeat And SOCS Box Containing 12 (e.g., chrX: 63444648-63446044), Iroquois Homeobox protein 4 (e.g., chr5: 1973342-1974948), adherens junctions associated protein 1 (e.g., chr1: 5035511-5037033), FA complementation group L (e.g., chr2: 59475581-59477094), Non-SMC Condensin II Complex Subunit D3 (e.g., chr11: 134034267-134035655), tribbles pseudokinase 2 (chr2: 12880585-12881771), or a combination thereof. In some aspects, the DMRs include chr1: 246058026-246059550, or a portion thereof. In some aspects, the DMRs include chr18: 76744751-76746907, or a portion thereof. In some aspects, the DMRs include chr10: 114073582-114075044, or a portion thereof. In some aspects, the DMRs include chr20: 2216856-2218239, or a portion thereof. In some aspects, the DMRs include chr2: 905862-907360, or a portion thereof. In some aspects, the DMRs include chr16: 79027537-79029698, or a portion thereof. In some aspects, the DMRs include chr17: 37756945-37758433, or a portion thereof. In some aspects, the DMRs include chr10: 44173499-44175059, or a portion thereof. In some aspects, the DMRs include chrX: 63444648-63446044, or a portion thereof. In some aspects, the DMRs include chr5: 1973342-1974948, or a portion thereof. In some aspects, the DMRs include chr1: 5035511-5037033, or a portion thereof. In some aspects, the DMRs include chr2: 59475581-59477094, or a portion thereof. In some aspects, the DMRs include chr11: 134034267-134035655, or a portion thereof. In some aspects, the DMRs include chr2: 12880585-12881771, or a portion thereof. In some aspects, the portion thereof is at least about 25%, at least about 50%, at least about 75%, or at least about 90% of the gene.

The methods of the present invention are generally applicable to animals. While many aspects of the present invention concern offspring ASD risk analysis by profiling male gametes (i.e., sperm), the methods may use genetic material from other sources, including female gametes, blood fractions, tissue homogenates, and other biofluids. In many aspects of the present invention, the subject is human. In some aspects, the subject is a prospective parent. In some aspects, semen is analyzed from a human male subject to determine risk of having an offspring with ASD. In some aspects, DNA is obtained from sperm in a semen sample from the subject.

The term “subject” as used herein refers to any individual or patient to which the disclosed methods are performed or from whom a biological material (e.g., sperm, a cell, or a biofluid) is obtained. Generally, the subject is human, although as will be appreciated by those in the art, the subject may be a non-human animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The subject may have a risk factor associated with having an offspring with ASD. A method may include identifying a subject at risk of having an offspring with ASD. ASD is associated with numerous familial and behavioral risk factors. Certain genetic conditions increase the risk for having offspring with ASD, including fragile X syndrome and tuberous sclerosis. Advanced parent age can also be a risk factor, with older parents having increased likelihood for having a child with ASD. Furthermore, incidences of ASD in a family often portends higher risk of ASD among siblings. Accordingly, in some aspects, the subject has a genetic ASD risk factor, is older than 35 years of age, is the parent of a child with ASD, or a combination thereof.

### Methods of Diagnosing Autism

Further disclosed herein are methods of diagnosing autism based on methylation in DNA obtained from semen samples. In certain aspects, the invention provides a method of diagnosing autism spectrum disorder (ASD) in a subject by measuring a DNA methylation status at one or more differentially methylated regions (DMRs) in a DNA sample from the subject; and determining a risk score based on DMRs methylation status, thereby diagnosing ASD in the subject.

In certain aspects, a difference in the DNA methylation status at one or more DMRs as compared to a control DNA methylation status is indicative of a higher likelihood of having ASD than a likelihood of having ASD as measured in the control DNA. In some aspects, a difference in the DNA methylation status at one or more DMRs as compared to a control DNA methylation status is indicative of a higher likelihood of having ASD than a likelihood of having ASD as measured in the control DNA. In some aspects, a difference in the DNA methylation status at one or more DMRs as compared to a control DNA methylation status is indicative of a higher likelihood of having ASD than a likelihood of having ASD as determined for the subject from which the control DNA was obtained. In some aspects, a differentially methylated region is characterized by variance in methylation density between a control sample and a sample from the subject. In some aspects, a difference in the DNA methylation status includes hypomethylation, hypermethylation or a combination thereof.

In some aspects, the DMRs are in genes selected from the group of genes set forth in Table 6, Table 7, or Table 8. In some aspects, the DMRs include a 5′ untranslated region (UTR). In some cases, the DMRs include a 3′ UTR. In some aspects, the DMRs include an intron. In some cases, the DMRs include an exon. In some aspects, the genes include an ASD-associated gene. In some cases, the genes are ASD-associated genes. In some aspects, the genes include a non-ASD-associated genes. In some aspects, the genes are non-ASD-associated genes. In some aspects, the DNA methylation status at the one or more DMRs is associated with an SRS score in the subject.

In some aspects, DNA methylation status is measured in at least 1, at least 2, at least 3, at least 5, at least 8, at least 12, at least 15, at least 20, at least 25, at least 30, at least 40, at least 50, at least 75, at least 100, at least 150, at least 200, or at least 250 DMRs. In some aspects, DNA methylation status is measured in at most 250, at most 200, at most 150, at most 100, at most 75, at most 50, at most 40, at most 30, at most 25, at most 20, at most 15, at most 12, at most 8, at most 5, or at most 3 DMRs. In some aspects, DNA methylation status is measured in about 3 to 10, in about 3 to 15, in about 5 to 15, in about 5 to 25, in about 8 to 30, in about 12 to 50, in about 20 to 50, in about 20 to 100, in about 50 to 100, or in about 100 to 500 DMRs.

In some cases, the DMRs are in genes selected from the group of genes set forth in Table 6. In some cases, the DMRs include at least 1, at least 2, at least 3, at least 4, at least 5, at least 6, at least 7, at least 8, at least 9, at least 10, at least 11, at least 12, at least 13, or all 14 genes in Table 6. In some cases, the DMRs include a gene associated with synaptic function, neurogenesis, development, or a combination thereof. In some cases, the DMRs include a gene or a 5′ or 3′ untranslated region of a gene associated with WW domain-containing oxidoreductase, RNA binding fox-1 homolog 1, spalt like transcription factor 3, Adenosine Deaminase RNA Specific B2, phosphofructokinase, zinc finger protein 536, vascular endothelial growth factor C, galanin receptor 1, RNA binding fox-1 homolog, junctional Adhesion Molecule 3, or a combination thereof. In some aspects, the DMRs include chr16: 79027119-79030054, or a portion thereof. In some aspects, the DMRs include chr16: 7065694-7068381, or a portion thereof. In some aspects, the DMRs include chr18: 76744751-76746907, or a portion thereof. In some aspects, the DMRs include chr16: 78376735-78378445. In some aspects, the DMRs include chr10: 1894909-1896546, or a portion thereof. In some aspects, the DMRs include chr16: 78974715-78976580, or a portion thereof. In some aspects, the DMRs include chr10: 3057233-3058630. In some aspects, the DMRs include chr19: 30745777-30747506, or a portion thereof. In some aspects, the DMRs include chr4: 177804604-177805652. In some aspects, the DMRs include chr18: 75687429-75689009, or a portion thereof. In some aspects, the DMRs include chr16: 6334658-6336272, or a portion thereof. In some aspects, the DMRs include chr11: 133989860-133991280, or a portion thereof. In some aspects, the DMRs include chr14: 99254155-99255193, or a portion thereof. In some aspects, the DMRs include chrX: 103301728-103303214, or a portion thereof. In some aspects, the portion thereof is at least about 25%, at least about 50%, at least about 75%, or at least about 90% of the gene. In some cases, the DMRs are between about 200 and 10000 nucleotides in length, between about 200 and 1500 nucleotides in length, between about 500 and 2500 nucleotides in length, between about 1000 and 5000 nucleotides in length, or between about 2000 and 10000 nucleotides in length.

In some cases, the DMRs are in genes selected from the group of genes set forth in Table 8. In some cases, the DMRs include at least 1, at least 2, at least 3, at least 4, at least 5, at least 6, at least 7, at least 8, at least 9, at least 10, at least 11, at least 12, at least 13, or all 14 genes in Table 8. In some cases, the DMRs include a gene associated with epigenetic regulation, embryonic development, cellular differentiation, neuronal signaling, or a combination thereof. In some cases, the DMRs include a gene or a 5′ or 3′ untranslated region of a gene associated with SET and MYND domain-containing protein 3, spalt like transcription factor 3, transglutaminase 3, WW Domain Containing Oxidoreductase, neuronal differentiation 2, zinc finger protein 32, Ankyrin Repeat And SOCS Box Containing 12, Iroquois Homeobox protein 4, adherens junctions associated protein 1, FA complementation group L, Non-SMC Condensin II Complex Subunit D3, tribbles pseudokinase 2, or a combination thereof. In some aspects, the DMRs include chr1: 246058026-246059550, or a portion thereof. In some aspects, the DMRs include chr18: 76744751-76746907, or a portion thereof. In some aspects, the DMRs include chr10: 114073582-114075044, or a portion thereof. In some aspects, the DMRs include chr20: 2216856-2218239, or a portion thereof. In some aspects, the DMRs include chr2: 905862-907360, or a portion thereof. In some aspects, the DMRs include chr16: 79027537-79029698, or a portion thereof. In some aspects, the DMRs include chr17: 37756945-37758433, or a portion thereof. In some aspects, the DMRs include chr10: 44173499-44175059, or a portion thereof. In some aspects, the DMRs include chrX: 63444648-63446044, or a portion thereof. In some aspects, the DMRs include chr5: 1973342-1974948, or a portion thereof. In some aspects, the DMRs include chr1: 5035511-5037033, or a portion thereof. In some aspects, the DMRs include chr2: 59475581-59477094, or a portion thereof. In some aspects, the DMRs include chr11: 134034267-134035655, or a portion thereof. In some aspects, the DMRs include chr2: 12880585-12881771, or a portion thereof. In some aspects, the portion thereof is at least about 25%, at least about 50%, at least about 75%, or at least about 90% of the gene.

In many aspects of the present invention, the subject is human. The subject may have a risk factor for ASD. In some cases, the subject has a genetic ASD risk factor. In some cases, the subject was conceived by a parent older than about 30, 35, or 40 years of age. In some cases, the subject was conceived by a parent who smoked before or during pregnancy. In some cases, the subject has a sibling with ASD. In some cases, the subject has an SRS score of at least about 60, at least about 70, at least about 80, at least about 90, at least about 100, at least about 110, or at least about 120. The sample can be a biofluid obtained from subject, such as a cell lysate or tissue homogenate. As non-limiting examples, DNA can be obtained from semen, plasma, serum, cerebrospinal fluid, synovial fluid, skin, lung lavage, sweat, crevicular fluid, bronchial lavage, tissue homogenates, cell culture samples, or a combination of sources thereof. In some aspects, DNA is obtained from sperm in a semen sample from the subject.

Further aspects of the present invention provide a method of determining an association between environmental factors and ASD risk. Changes within methylation status within the DMRs disclosed herein can indicate a change in risk for developing or having an offspring with ASD. Leveraging this discovery, a method of determining an association between exposure to an environmental factor in a subject and an increased risk of having an offspring with autism spectrum disorder (ASD) can include measuring a first DNA methylation status at differentially methylated regions (DMRs) in DNA from a first semen sample from the subject prior to exposure to the environmental factor; measuring a second DNA methylation status at DMRs in DNA from a second semen sample from the subject after to exposure to the environmental factor; and comparing the first and the second methylation status at the DMRs; thereby determining an association between exposure to an environmental factor and an increased risk of having an offspring with ASD. In this method, a change in the methylation status at DMRs between the first DNA methylation status and the second DNA methylation status can be indicative of an association between the environmental factor and an increased risk of having an offspring with autism ASD. Alternatively, or in addition thereto, a change in the methylation status at DMRs between the first DNA methylation status and the second DNA methylation status can be indicative of an association between the environmental factor and a decreased risk of having an offspring with autism ASD (e.g., decreased risk for having an offspring with autism upon improved diet or cessation of smoking).

In many aspects of the present invention, the subject is human. In some aspects, the subject is a prospective parent. In some aspects, semen is analyzed from a human male subject to determine risk of having an offspring with ASD. In some aspects, DNA is obtained from sperm in a semen sample from the subject.

In some aspects, the DMRs are in genes selected from the group of genes set forth in Table 6, Table 7, or Table 8. In some aspects, the DMRs include a 5′ untranslated region (UTR). In some cases, the DMRs include a 3′ UTR. In some aspects, the DMRs include an intron. In some cases, the DMRs include an exon. In some aspects, the genes include an ASD-associated gene. In some cases, the genes are ASD-associated genes. In some aspects, the genes include a non-ASD-associated genes. In some aspects, the genes are non-ASD-associated genes. In some aspects, the DNA methylation status at the one or more DMRs is associated with an SRS score in the subject.

In some aspects, DNA methylation status is measured in at least 3, at least 5, at least 8, at least 12, at least 15, at least 20, at least 25, at least 30, at least 40, at least 50, at least 75, at least 100, at least 150, at least 200, or at least 250 DMRs. In some aspects, DNA methylation status is measured in at most 250, at most 200, at most 150, at most 100, at most 75, at most 50, at most 40, at most 30, at most 25, at most 20, at most 15, at most 12, at most 8, at most 5, or at most 3 DMRs. In some aspects, DNA methylation status is measured in about 3 to 10, in about 3 to 15, in about 5 to 15, in about 5 to 25, in about 8 to 30, in about 12 to 50, in about 20 to 50, in about 20 to 100, in about 50 to 100, or in about 100 to 500 DMRs.

In some cases, the DMRs are in genes selected from the group of genes set forth in Table 6. In some cases, the DMRs include at least 1, at least 2, at least 3, at least 4, at least 5, at least 6, at least 7, at least 8, at least 9, at least 10, at least 11, at least 12, at least 13, or all 14 genes in Table 6. In some cases, the DMRs include a gene associated with synaptic function, neurogenesis, development, or a combination thereof. In some cases, the DMRs include a gene or a 5′ or 3′ untranslated region of a gene associated with WW domain-containing oxidoreductase, RNA binding fox-1 homolog 1, spalt like transcription factor 3, Adenosine Deaminase RNA Specific B2, phosphofructokinase, zinc finger protein 536, vascular endothelial growth factor C, galanin receptor 1, RNA binding fox-1 homolog, junctional Adhesion Molecule 3, or a combination thereof. In some aspects, the DMRs include chr16: 79027119-79030054, or a portion thereof. In some aspects, the DMRs include chr16: 7065694-7068381, or a portion thereof. In some aspects, the DMRs include chr18: 76744751-76746907, or a portion thereof. In some aspects, the DMRs include chr16: 78376735-78378445. In some aspects, the DMRs include chr10: 1894909-1896546, or a portion thereof. In some aspects, the DMRs include chr16: 78974715-78976580, or a portion thereof. In some aspects, the DMRs include chr10: 3057233-3058630. In some aspects, the DMRs include chr19: 30745777-30747506, or a portion thereof. In some aspects, the DMRs include chr4: 177804604-177805652. In some aspects, the DMRs include chr18: 75687429-75689009, or a portion thereof. In some aspects, the DMRs include chr16: 6334658-6336272, or a portion thereof. In some aspects, the DMRs include chr11: 133989860-133991280, or a portion thereof. In some aspects, the DMRs include chr14: 99254155-99255193, or a portion thereof. In some aspects, the DMRs include chrX: 103301728-103303214, or a portion thereof. In some aspects, the portion thereof is at least about 25%, at least about 50%, at least about 75%, or at least about 90% of the gene.

In some cases, the DMRs are in genes selected from the group of genes set forth in Table 8. In some cases, the DMRs include at least 1, at least 2, at least 3, at least 4, at least 5, at least 6, at least 7, at least 8, at least 9, at least 10, at least 11, at least 12, at least 13, or all 14 genes in Table 8. In some cases, the DMRs include a gene associated with epigenetic regulation, embryonic development, cellular differentiation, neuronal signaling, or a combination thereof. In some cases, the DMRs include a gene or a 5′ or 3′ untranslated region of a gene associated with SET and MYND domain-containing protein 3, spalt like transcription factor 3, transglutaminase 3, WW Domain Containing Oxidoreductase, neuronal differentiation 2, zinc finger protein 32, Ankyrin Repeat And SOCS Box Containing 12, Iroquois Homeobox protein 4, adherens junctions associated protein 1, FA complementation group L, Non-SMC Condensin II Complex Subunit D3, tribbles pseudokinase 2, or a combination thereof. In some aspects, the DMRs include chr1: 246058026-246059550, or a portion thereof. In some aspects, the DMRs include chr18: 76744751-76746907, or a portion thereof. In some aspects, the DMRs include chr10: 114073582-114075044, or a portion thereof. In some aspects, the DMRs include chr20: 2216856-2218239, or a portion thereof. In some aspects, the DMRs include chr2: 905862-907360, or a portion thereof. In some aspects, the DMRs include chr16: 79027537-79029698, or a portion thereof. In some aspects, the DMRs include chr17: 37756945-37758433, or a portion thereof. In some aspects, the DMRs include chr10: 44173499-44175059, or a portion thereof. In some aspects, the DMRs include chrX: 63444648-63446044, or a portion thereof. In some aspects, the DMRs include chr5: 1973342-1974948, or a portion thereof. In some aspects, the DMRs include chr1: 5035511-5037033, or a portion thereof. In some aspects, the DMRs include chr2: 59475581-59477094, or a portion thereof. In some aspects, the DMRs include chr11: 134034267-134035655, or a portion thereof. In some aspects, the DMRs include chr2: 12880585-12881771, or a portion thereof. In some aspects, the portion thereof is at least about 25%, at least about 50%, at least about 75%, or at least about 90% of the gene.

In many aspects of the present invention, the subject is human. The subject may have a risk factor for ASD. In some cases, the subject has a genetic ASD risk factor. In some cases, the subject was conceived by a parent older than about 30, 35, or 40 years of age. In some cases, the subject has a sibling with ASD. In some cases, the subject has an SRS score of at least about 60, at least about 70, at least about 80, at least about 90, at least about 100, at least about 110, or at least about 120.

Numerous DNA methylation detection platforms are known in the art and are applicable to the methods disclosed herein. In some aspects, DNA methylation status is measured with methylation specific PCR, bisulfite sequencing, capture bisulfite sequencing, whole genome bisulfite sequencing, pyrosequencing, single-strand conformation polymorphism (SSCP) analysis, restriction analysis, microarray technology, including bead microarray technology, or proteomics. Methylation-sensitive DNA sequencing methods typically provide quantitative DNA methylation statuses, for example regional (e.g., gene-specific or CpG island-specific)methylation density, site-specific methylation frequency, or methylation frequency ratios relative to a standard.

For many of the methods disclosed herein, measuring DNA methylation status includes the use of a high-throughput array such as comprehensive high-throughput array-based relative methylation (CHARM, e.g., as detailed in Irizarry et al. Genome Res., 2008, 18 (5): 780-790.). CHARM utilizes methylation specific DNA digestion to identify site-specific methylation. Typically, a first fraction of DNA subjected to methylation specific digestion (e.g., with McrBC) is compared to a second fraction of DNA not subjected to the methylation specific digestion. Methylated sequences depleted in the digested fraction are then quantified to determine methylation site and frequency.

In some aspects, measuring DNA methylation status includes bisulfite treatment. DNA treatment with bisulfite can selectively convert unmethylated cytosine to uracil while leaving methylated cytosine unchanged. The degree of cytosine to uracil conversion in a genomic region can be quantified, for example by methylation specific PCR, pyrosequencing, methylation-sensitive single-strand conformation analysis, high resolution melting analysis, methylation-sensitive single-nucleotide primer extension, uracil-sensitive cleavage (e.g., with an RNase), or by direct (e.g., nanopore) sequencing.

DNA methylation can also be measured by quantitative PCR (qPCR). Such methods can include methylated DNA enrichment with a methylation-sensitive DNA binding protein prior to amplification and quantitation. qPCR can also include a methylation-sensitive restriction enzyme which selectively cleaves methylated or unmethylated sites, enabling differential analysis of methylated and unmethylated DNA (e.g., Hpall tiny fragment enrichment by ligation-mediated PCR).

Aspects of the present invention provide a kit for performing a method of the present invention. In some aspects, the kit is for determining whether a subject has or is at risk of having or inheriting a risk of having autism spectrum disorder (ASD). The kit can include a reagent for determining a DNA methylation status at one or more differentially methylated regions (DMRs) in a DNA sample from the subject; and instructions for using the reagent.

In some aspects, the reagent includes an agent capable of selectively binding the one or more DMRs. As non-limiting examples, the probe can be an oligonucleotide, a primer, a primer pair, or a combination thereof. The probe can be configured to only bind to the DMR if its methylation is above or below a threshold level. Similarly, the probe can be configured to bind to the DMR following treatment with a methylation sensitive reagent, such as bisulfite. In some aspects, the probe includes a primer for amplifying at least a portion of the DMR.

In some aspects, the reagent includes an agent capable of cleaving DNA, such as a nuclease. The nuclease can be configured to generate DNA fragments of a target size range, such as about 1 to 5 kb. The nuclease can be configured to selectively cleave methylated DNA, such as at methylated CpGs.

In some aspects, the one or more DMRs are in genes selected from the group of genes set forth in Table 6, Table 7, and Table 8. In some cases, the kit includes reagents for determining a DNA methylation status for at least 1, at least 2, at least 3, at least 4, at least 5, at least 6, at least 7, at least 8, at least 9, at least 10, at least 11, at least 12, at least 13, or all 14 genes in Table 6. In some cases, the kit includes reagents for determining a DNA methylation status for at least 1, at least 2, at least 3, at least 4, at least 5, at least 6, at least 7, at least 8, at least 9, at least 10, at least 11, at least 12, at least 13, or all 14 genes in Table 8.

Presented below are examples discussing epigenetic changes in DMRs and their association with risk of developing ASD contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Methods

The relationship between paternal autistic traits and the sperm epigenome were associated with autistic traits in children at 36 months enrolled in the Early Autism Risk Longitudinal Investigation (EARLI) cohort. EARLI is a pregnancy cohort that recruited and enrolled pregnant women in the first half of pregnancy who already had a child with ASD. After maternal enrollment, EARLI fathers were approached and asked to provide a semen specimen. Participants were included in the present study if they had genotyping, sperm methylation data, and Social Responsiveness Scale (SRS) score data available. Using the CHARM array, genome-scale methylation analyses were performed on DNA from semen samples contributed by EARLI fathers. The SRS—a 65-item questionnaire measuring social communication deficits on a quantitative scale—was used to evaluate autistic traits in EARLI fathers (n=45) and children (n=31). 94 significant child SRS-associated differentially methylated regions (DMRs) and 14 significant paternal SRS-associated DMRs (fwer p<0.05) were identified. Many child SRS-associated DMRs were annotated to genes implicated in ASD and neurodevelopment. Six DMRs overlapped across the two outcomes (fwer p<0.1), and, 16 DMRs overlapped with previous child autistic trait findings at 12 months of age (fwer p<0.05). Child SRS-associated DMRs contained CpG sites independently found to be differentially methylated in postmortem brains of individuals with and without autism. These findings suggest paternal germline methylation is associated with autistic traits in three-year-old offspring. These prospective results for autism-associated traits, in a cohort with a family history of ASD, highlight the potential importance of sperm epigenetic mechanisms in autism.

### Study Sample

### The Early Autism Risk Longitudinal Investigation (EARLI) Study

EARLI enrolled pregnant women, who have had a child with ASD, during a subsequent pregnancy and prospectively followed that infant sibling from birth through 36 months of age. A detailed description of the EARLI study methods can be found in Newschaffer et al. (Journal of neurodevelopmental disorders. 2012; 4 (1): 7). Sperm samples were collected from EARLI fathers around the time of maternal enrollment during pregnancy. The EARLI study sample is racially, ethnically, and socioeconomically diverse. The EARLI study was reviewed and approved by Human Subjects Institutional Review Boards (IRBs) from each of the four study sites, and informed consent was obtained from all subjects.

### Phenotype Assessment

The Social Responsiveness Scale (SRS) is a 65-item questionnaire designed to measure an individual's social impairments and is often used as an early screener for autism. The SRS measures five behavioral subscales in individuals-social awareness, social information processing or cognition, the capacity for social communication, social anxiety and avoidant behaviors, and autistic preoccupations and traits The SRS has strong psychometric properties and high validity and reproducibility, and children with ASD diagnoses often have higher scores on the SRS. Evaluation with the preschool SRS was completed by mothers at 36-months for EARLI children, while paternal evaluations were completed by self-report using the adult version of the SRS. Total raw SRS scores are created by summing the scores of the coded items in the assessment. A raw SRS score <60 is considered normative, while higher SRS scores are associated with more autistic-like behaviors.

### Laboratory Analyses

### Sample Processing and DNA Extraction

Semen samples were frozen upon collection and shipped frozen with four −10° C. freezer packs directly to the Johns Hopkins Biological Repository (JHBR) for storage (−80° C.) until processing. Genomic DNA from semen samples was isolated via QIAgen QIAsymphony automated workstation with the Blood 1000 protocol of the DSP DNA Midi kit (Cat. No. 937255, Qiagen, Valencia, CA) as per manufacturer's instructions.

### CHARM DNA Methylation Measurement

Genome-scale sperm DNAm was measured using the Comprehensive High-throughput Arrays for Relative Methylation (CHARM) assay, and detailed protocols for sperm methylation measurements via CHARM are described in Feinberg et al 2015. Briefly, Genomic sperm DNA (4 μg) was sheared, digested with McrBC, gel-purified, labeled, and hybridized to arrays as described. Arrays include probes covering all annotated and non-annotated promoters and microRNA sites in addition to features present in the original CHARM method. The raw methylation data analyzed in the current study was previously uploaded to the National Database for Autism Research (NDAR) study 377.

### Illumina 450k DNA Methylation Measurement

Detailed protocols for sperm methylation measurements via the Illumina Infinium HumanMethylation450 BeadChip assay (referred to as 450K) are as described in Feinberg et al 2015. DNA methylation was measured for a subset of available sperm samples via the 450k array. (Illumina, San Diego, CA). Genomic DNA (lug) was processed by the Johns Hopkins University SNP Center using the automated Infinium workflow.

### Genotyping for Ancestry Principal Components

DNA from buffy coat, white blood cells, and saliva were run on the Affymetrix omni5 exome array at the Johns Hopkins University SNP center for genotyping analysis. Using PLINK v1.90 parents were subset from the EARLI dataset and merged with the 1000G Phase3 v5 reference keeping only overlapping SNPs and a MAF filter of ≥0.05. Principal components (PCs) 1-10 were then assembled using smartpca from EIGENSOFT 6.1.4 using 1000G Phase3 v5 as an anchor for ancestry. Ancestral principal components were used in downstream statistical analyses to adjust for genetic ancestry, controlling for any methylation changes that might result from differences in population stratification.

### Statistical Analyses

### Bivariate Summary Statistics

Fathers were included in the present study if they had genotyping, sperm methylation data, SRS score, and covariate data available (n=45 for fathers, n=31 for offspring). Offspring were included if they had paternal SRS scores, paternal sperm DNA methylation, their own SRS scores, and covariate data available (n=31). Not all children whose fathers had methylation data also had SRS scores, leading to the differences in sample size for these analyses. Differences in SRS scores across demographic variables and correlations between paternal and offspring SRS outcomes were assessed using t-tests, ANOVA, MWW rank-sum tests, and Spearman rank correlations, where appropriate. Similar tests were also performed to assess the degree of association between estimated surrogate variables (described below) and demographic variables.

### DNA Methylation Data Processing

CHARM raw data were pre-processed as previously described8 using the CHARM package (v.2.8.0) in R (version 3.0.3). Briefly, probe-level percentage DNA methylation estimates were obtained by first removing background signal, followed by normalization using control probes. Following normalization, we excluded background, control, and repetitive probe groups, resulting in 3,811,046 total probes per array for each sample. Illumina Infinium methylation data for the overlapping subjects was processed in R version 3.4.0 using the preprocessNoob function in the minfi package (v 1.22.1). No probes were excluded during preprocessing.

### Surrogate Variable Analysis

Surrogate variable analysis (SVA) was performed on percentage methylation estimates as described in Feinberg et al 2015. SVA was used to estimate latent factors or batch effects that may influencing DNA methylation. We estimated the number of surrogate variables (SVs) to include in statistical models using the Buja and Eyuboglu (“be”) algorithm, which quantifies latent variables present in data. SVs are then adjusted for as confounders in downstream differential methylation analyses. R version 4.1 was used for running the SVA R package and for performing all other downstream analyses unless otherwise specified.

### Identification of Outcome-Associated Changes in Regional DNA Methylation

Methods for identifying regions of CHARM DNAm that were associated with SRS scores are described in detail in Feinberg et al 2015. Briefly, we used the “bump hunting” approach previously developed for CHARM, adjusting for estimated SVs as well as any potential confounders. The statistical model for the paternal SRS analysis treated SRS as the outcome of interest and included 9 SVs and 4 paternal ancestry PCs. This analysis included the 45 fathers that met the study inclusion criteria mentioned above. The statistical model for the child SRS score treated SRS as the outcome of interest and included 5 SVs, 4 paternal ancestry PCs, child's sex, and paternal education. This analysis included just the 31 father-child pairs. DMRs were identified by smoothing the linear effects, and thresholding smoothed statistics across all probes (cutoff=99.9th percentile). P-values were calculated for each DMR from a genome-wide empirical distribution of null statistics generated using a linear model bootstrapping approach across 10,000 permutations. Significant DMRs had a genome-wide family-wise error rate (FWER) p<0.05.

### Cross-Platform Validation

Cross-validation and data quality assessment for the Illumina 450K methylation data are is described in detail in Feinberg et al 2015. In summary, we attempted to validate DMRs discovered via CHARM score using overlapping genomic coverage on the 450K array in a partially overlapping set of sperm samples. At the CpG sites covered by both arrays, linear regression was first used to test the relationship between single-site 450K DNA methylation and paternal or 36-month outcome SRS scores. Statistical models were adjusted for surrogate variables estimated from the data of only overlapping samples (n=29 samples with paternal SRS; n=21 samples with child SRS). Spearman correlation tests were then used to calculate the correlation between effect estimates from CHARM and 450K.

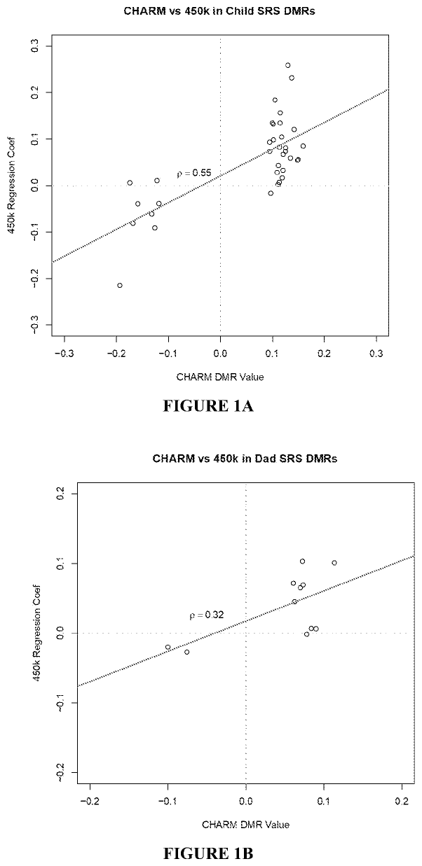

Feinberg et al 2015 previously reported the data quality assessment for the Illumina 450K methylation data. Table 1 shows the degree of association between 450K variables of each of the estimated surrogate variables for child SRS; Table 2 shows the degree of association between 450K variables of each of the estimated surrogate variables for paternal SRS. Among the DMRs that were significantly associated with child SRS scores from the CHARM array, we extracted probes from the Illumina 450K array that were located within 500 base pairs (bp) of the CHARM DMR boundaries. This was feasible for 35 (37.2%) of the 94 CHARM-identified DMRS. The direction of association between child SRS and DNA methylation was consistent for 30 (85.7%) of the 35 regions (rho-0.49, FIG. 1A). Among the 14 DMRs that were significantly associated with paternal SRS scores from the CHARM array we again extracted probes from the 450K array that were located within 500 bp of the CHARM DMR boundaries, which was feasible for 11 (78.6%) of the 14 regions. The direction of the association between paternal SRS and DNA methylation was consistent for 10 (90.9%) of the 11 regions (rho=0.32, FIG. 1B).

TABLE 1
Child SRS 450K SV p value associations

| | SV1 | SV2 | SV3 | SV4 |
|---|---|---|---|---|
| Hyb.date | 0.895 | 0.0879 | 0.0125 | 0.826 |
| Image.date | 0.88 | 0.676 | 0.264 | 0.841 |
| Plate.ID | 0.109 | 0.74 | 0.0124 | 0.361 |
| Sample.Well | 0.803 | 0.107 | 0.269 | 0.42 |
| Slide.ID | 0.301 | 0.226 | 0.121 | 0.286 |
| Array.ID | 0.452 | 0.258 | 0.663 | 0.248 |
| ArrayRow6 | 0.835 | 0.287 | 0.47 | 0.405 |
| ExtractionLab | 0.0977 | 0.587 | 0.147 | 0.211 |
| Subject.Type | 0.272 | 0.313 | 0.883 | 0.595 |
| Site | 0.313 | 0.665 | 0.363 | 0.785 |
| Race_EARLImar | 0.189 | 0.0278 | 0.0676 | 0.831 |
| EverSmoke | 0.173 | 0.64 | 0.0315 | 0.943 |
| PregSib.Sex | 0.104 | 0.535 | 0.549 | 0.622 |
| DadEdu | 0.428 | 0.49 | 0.822 | 0.938 |
| Age | 0.658 | 0.346 | 0.144 | 0.0634 |
| Gest.Age.Weeks | 0.891 | 0.459 | 0.306 | 0.895 |
| SRS_age_Sib_36mos | 0.903 | 0.755 | 0.748 | 0.274 |
| Weeks.before.Birth | 0.0706 | 0.266 | 0.579 | 0.257 |
| bw_g | 0.772 | 0.222 | 0.883 | 0.77 |
| SRS_Raw | 0.116 | 0.48 | 0.749 | 0.231 |
| logSRS | 0.22 | 0.459 | 0.978 | 0.575 |
| Race.PC1 | 0.0648 | 0.0255 | 0.0579 | 0.577 |
| Race.PC2 | 0.59 | 0.0485 | 0.174 | 0.73 |
| Race.PC3 | 0.114 | 0.786 | 0.592 | 0.725 |
| Race.PC4 | 0.509 | 0.314 | 0.819 | 0.954 |
| Race.PC5 | 0.668 | 0.674 | 0.742 | 0.969 |
| Race.PC6 | 0.702 | 0.769 | 0.644 | 0.56 |
| Race.PC7 | 0.091 | 0.123 | 0.0325 | 0.662 |
| Race.PC8 | 0.972 | 0.488 | 0.498 | 0.883 |
| Race.PC9 | 0.636 | 0.32 | 0.94 | 0.702 |
| Race.PC10 | 0.674 | 0.473 | 0.605 | 0.407 |

TABLE 2
Paternal SRS 450K SV p value association

| | SV1 | SV2 | SV3 | SV4 | SV5 | SV6 |
|---|---|---|---|---|---|---|
| Hyb.date | 0.818 | 0.0378 | 0.00725 | 0.957 | 0.799 | 0.514 |
| Image.date | 0.909 | 0.448 | 0.187 | 0.665 | 0.901 | 0.767 |
| Plate.ID | 0.436 | 0.454 | 0.000148 | 0.508 | 0.615 | 0.218 |
| Sample.Well | 0.802 | 0.174 | 0.859 | 0.55 | 0.87 | 0.678 |
| Slide.ID | 0.413 | 0.173 | 0.264 | 0.277 | 0.724 | 0.496 |
| Array.ID | 0.453 | 0.641 | 0.867 | 0.488 | 0.631 | 0.35 |
| ArrayRow6 | 0.824 | 0.698 | 0.534 | 0.369 | 0.253 | 0.956 |
| ExtractionLab | 0.572 | 0.853 | 0.854 | 0.0112 | 0.259 | 0.15 |
| Subject.Type | 0.364 | 0.476 | 0.477 | 0.863 | 0.0759 | 0.595 |
| Site | 0.535 | 0.915 | 0.574 | 0.353 | 0.0486 | 0.81 |
| Race_EARLImar | 0.332 | 0.0383 | 0.0775 | 0.179 | 0.5 | 0.74 |
| EverSmoke | 0.231 | 0.666 | 0.12 | 0.659 | 0.29 | 0.903 |
| PregSib.Sex | 0.46 | 0.364 | 0.488 | 0.857 | 0.23 | 0.401 |
| DadEdu | 0.351 | 0.845 | 0.865 | 0.926 | 0.0282 | 0.88 |
| Age | 0.383 | 0.204 | 0.428 | 0.0106 | 0.0262 | 0.727 |
| SRS.Age.Dad | 0.298 | 0.207 | 0.44 | 0.00764 | 0.0191 | 0.766 |
| Gest.Age.Weeks | 0.636 | 0.353 | 0.526 | 0.806 | 0.647 | 0.627 |
| Weeks.before.Birth | 0.283 | 0.0831 | 0.131 | 0.403 | 0.666 | 0.431 |
| bw_g | 0.898 | 0.486 | 0.377 | 0.485 | 0.851 | 0.375 |
| SRS.dad | 0.201 | 0.528 | 0.171 | 0.588 | 0.16 | 0.121 |
| logSRS.dad | 0.593 | 0.457 | 0.27 | 0.863 | 0.256 | 0.0403 |
| Race.PC1 | 0.0756 | 0.0142 | 0.0867 | 0.0281 | 0.809 | 0.567 |
| Race.PC2 | 0.605 | 0.0948 | 0.952 | 0.215 | 0.251 | 0.418 |
| Race.PC3 | 0.683 | 0.679 | 0.326 | 0.933 | 0.0141 | 0.702 |
| Race.PC4 | 0.743 | 0.394 | 0.484 | 0.704 | 0.467 | 0.345 |
| Race.PC5 | 0.919 | 0.506 | 0.374 | 0.778 | 0.856 | 0.262 |
| Race.PC6 | 0.28 | 0.623 | 0.393 | 0.595 | 0.37 | 0.168 |
| Race.PC7 | 0.342 | 0.0538 | 0.69 | 0.372 | 0.166 | 0.797 |
| Race.PC8 | 0.48 | 0.253 | 0.263 | 0.918 | 0.978 | 0.357 |
| Race.PC9 | 0.984 | 0.25 | 0.313 | 0.463 | 0.727 | 0.41 |
| Race.PC10 | 0.885 | 0.302 | 0.493 | 0.351 | 0.929 | 0.203 |

Comparison with Independent Datasets

### AOSI

Significant DMRs associated with child SRS scores were compared to previous findings of DMRs associated with AOSI scores in 12-month-old EARLI participants. The GenomicRanges Bioconductor package was used to determine DMRs in common between the two datasets.

### Autism Brain Data

Comparison with autism brain data is described in detail in Feinberg et al 2015. In short, we downloaded publicly available Illumina 450K data (GSE53162) from post-mortem human brain tissues from individuals with ASD (n=19) and controls (n=21). Methylation data was available for the prefrontal cortex, temporal cortex, and cerebellum from this dataset. Data were normalized as described in Feinberg et al 2015, and mean methylation differences were calculated between ASD cases and controls using the limma Bioconductor package. Sites that were significantly differentially methylated (at p<0.05) were compared to the child SRS-associated DMR list to see if there were any commonalities using the GenomicRanges Bioconductor package.

### SFARI

The Simons Foundation for Autism Research Initiative (SFARI) has a publicly available list of 1,231 genes they have identified in the literature as being associated with autism. The Simons Foundation has a gene scoring system that scrutinizes all available evidence that might support a gene's relevance to ASD risk, and subsequently categorizes those genes in a way that reflects the strength of the evidence that a gene may confer ASD risk. This information has been compiled into the SFARI Gene database, and we used this dataset to determine whether there were overlaps with genes associated with child SRS-associated DMRs.

### Example 2

### Results

### Gene Enrichment Analysis

Gene ontology (GO) analysis was performed as previously described in Feinberg et al 2015. We tested for enrichment of genes within 10 kb of DMRs with FWER p<0.1 based on Gene Ontology Biological Process database, using the hypergeometric test restricted to gene sets with at least four members. We used the GOstats R Bioconductor package to compare genes mapped to DMRs (FWER p<0.1) to all genes on the CHARM array with an Entrez ID as background.

### Study Sample Characteristics

### Methylation Data Quality Assessment

Methylation measurement quality did not differ by outcome. Neither child nor paternal SRS scores varied by CHARM DNA shearing date, hybridization date, shearing matching, CHARM gel, or gel location. Table 3 shows the degree of association between CHARM variables of each of the estimated surrogate variables for child SRS; Table 4 shows the degree of association between CHARM variables of each of the estimated surrogate variables for paternal SRS.

TABLE 3
Child SRS CHARM SV p value associations

| | SV1 | SV2 | SV3 | SV4 | SV5 |
|---|---|---|---|---|---|
| Site | 0.738 | 0.537 | 0.414 | 0.294 | 0.543 |
| PregSib.Sex | 0.291 | 0.188 | 0.691 | 0.257 | 0.567 |
| Dx | 0.131 | 0.72 | 0.643 | 0.0442 | 0.204 |
| Subject.Type | 0.358 | 0.342 | 0.791 | 0.9 | 0.253 |
| EverSmoke | 0.824 | 0.00305 | 0.318 | 0.942 | 0.297 |
| HybDate | 0.147 | 0.00199 | 0.259 | 0.0199 | 0.511 |
| Date.Sheared | 0.0617 | 0.0305 | 0.0819 | 0.43 | 0.181 |
| HS. | 0.217 | 0.0275 | 0.562 | 0.114 | 0.484 |
| RubyID | 0.217 | 0.0275 | 0.562 | 0.114 | 0.484 |
| CHARM.Gel.ID | 0.016 | 0.0279 | 0.0546 | 0.402 | 0.0275 |
| Gel.Location | 0.834 | 0.767 | 0.572 | 0.78 | 0.834 |
| DadEdu | 0.0956 | 0.569 | 0.945 | 0.463 | 0.438 |
| Race_EARLImar | 0.84 | 0.512 | 0.769 | 0.299 | 0.511 |
| Age | 0.585 | 0.0165 | 0.82 | 0.61 | 0.758 |
| Gest.Age.Weeks | 0.176 | 0.0993 | 0.445 | 0.949 | 0.862 |
| bw_g | 0.632 | 0.0477 | 0.128 | 1 | 0.993 |
| AOSI_12mos | 0.199 | 0.549 | 0.0055 | 0.107 | 0.884 |
| SRS | 0.0359 | 0.105 | 0.767 | 0.791 | 0.693 |
| logSRS | 0.0454 | 0.145 | 0.712 | 0.879 | 0.828 |
| Race.PC1 | 0.701 | 0.38 | 0.345 | 0.438 | 0.282 |
| Race.PC2 | 0.4 | 0.88 | 0.417 | 0.115 | 0.158 |
| Race.PC3 | 0.643 | 0.621 | 0.625 | 0.642 | 0.785 |
| Race.PC4 | 0.472 | 0.921 | 0.65 | 0.146 | 0.302 |
| Race.PC5 | 0.255 | 0.738 | 0.687 | 0.353 | 0.602 |
| Race.PC6 | 0.502 | 0.169 | 0.9 | 0.441 | 0.144 |
| Race.PC7 | 0.131 | 0.159 | 0.347 | 0.444 | 0.583 |
| Race.PC8 | 0.67 | 0.77 | 0.642 | 0.56 | 0.681 |
| Race.PC9 | 0.853 | 0.942 | 0.485 | 0.212 | 0.483 |
| Race.PC10 | 0.236 | 0.493 | 0.955 | 0.985 | 0.852 |

TABLE 4
Paternal SRS CHARM SV p value association

| | SV1 | SV2 | SV3 | SV4 | SV5 | SV6 | SV7 | SV8 | SV9 |
|---|---|---|---|---|---|---|---|---|---|
| Site | 0.727 | 0.796 | 0.919 | 0.828 | 0.821 | 0.405 | 0.184 | 0.0299 | 0.42 |
| PregSib.Sex | 0.0699 | 0.833 | 0.964 | 0.253 | 0.38 | 0.104 | 0.719 | 0.0427 | 0.54 |
| Dx | 0.32 | 0.731 | 0.273 | 0.937 | 0.309 | 0.478 | 0.438 | 0.809 | 0.619 |
| Subject.Type | 0.838 | 0.529 | 0.343 | 0.948 | 0.55 | 0.337 | 0.0485 | 0.806 | 0.627 |
| EverSmoke | 0.255 | 0.296 | 0.12 | 0.234 | 0.51 | 0.808 | 0.361 | 0.796 | 0.0519 |
| HybDate | 0.0189 | 0.0889 | 0.00804 | 0.414 | 0.000766 | 0.354 | 0.0538 | 0.358 | 0.153 |
| Date.Sheared | 0.0188 | 0.0899 | 0.018 | 0.103 | 0.519 | 0.000675 | 0.0196 | 0.115 | 0.768 |
| HS. | 0.762 | 0.122 | 0.161 | 0.987 | 0.226 | 0.736 | 0.973 | 0.574 | 0.397 |
| RubyID | 0.762 | 0.122 | 0.161 | 0.987 | 0.226 | 0.736 | 0.973 | 0.574 | 0.397 |
| CHARM.Gel.ID | 0.152 | 0.179 | 0.0352 | 0.804 | 0.000834 | 0.00104 | 0.0218 | 0.0686 | 0.274 |
| Gel.Location | 0.817 | 0.846 | 0.155 | 0.859 | 0.889 | 0.796 | 0.684 | 0.853 | 0.454 |
| DadEdu | 0.186 | 0.66 | 0.932 | 0.586 | 0.939 | 0.272 | 0.708 | 0.00124 | 0.312 |
| Race_EARLImar | 0.668 | 0.357 | 0.652 | 0.0618 | 0.54 | 0.153 | 0.849 | 0.115 | 0.258 |
| Age | 0.222 | 0.0566 | 0.0219 | 0.768 | 0.755 | 0.225 | 0.79 | 0.759 | 0.309 |
| SRS.Age.Dad | 0.19 | 0.0627 | 0.018 | 0.82 | 0.813 | 0.176 | 0.747 | 0.713 | 0.262 |
| Gest.Age.Weeks | 0.638 | 0.985 | 0.805 | 0.396 | 0.255 | 0.944 | 0.566 | 0.246 | 0.567 |
| bw_g | 0.798 | 0.625 | 0.271 | 0.737 | 0.171 | 0.391 | 0.989 | 0.705 | 0.784 |
| AOSI_12 mos | 0.219 | 0.0187 | 0.14 | 0.508 | 0.438 | 0.725 | 0.665 | 0.552 | 0.494 |
| SRS | 0.0612 | 0.37 | 0.21 | 0.838 | 0.929 | 0.0995 | 0.995 | 0.655 | 0.437 |
| SRS.dad | 0.752 | 0.16 | 0.281 | 0.309 | 0.489 | 0.685 | 0.553 | 0.0432 | 0.222 |
| logSRS.dad | 0.621 | 0.0775 | 0.0967 | 0.498 | 0.366 | 0.775 | 0.982 | 0.0241 | 0.496 |
| Race.PC1 | 0.607 | 0.106 | 0.468 | 0.893 | 0.62 | 0.0334 | 0.894 | 0.11 | 0.862 |
| Race.PC2 | 0.473 | 0.203 | 0.137 | 0.345 | 0.207 | 0.0891 | 0.414 | 0.614 | 0.247 |
| Race.PC3 | 0.877 | 0.682 | 0.53 | 0.0288 | 0.895 | 0.211 | 0.771 | 0.802 | 0.43 |
| Race.PC4 | 0.477 | 0.242 | 0.186 | 0.504 | 0.154 | 0.53 | 0.316 | 0.67 | 0.208 |
| Race.PC5 | 0.0644 | 0.465 | 0.465 | 0.602 | 0.077 | 0.524 | 0.0594 | 0.649 | 0.97 |
| Race.PC6 | 0.185 | 0.83 | 0.056 | 0.394 | 0.154 | 0.397 | 0.0259 | 0.658 | 0.711 |
| Race.PC7 | 0.251 | 0.0552 | 0.635 | 0.775 | 0.826 | 0.259 | 0.0905 | 0.426 | 0.197 |
| Race.PC8 | 0.239 | 0.439 | 0.292 | 0.861 | 0.264 | 0.357 | 0.0142 | 0.682 | 0.859 |
| Race.PC9 | 0.277 | 0.137 | 0.133 | 0.869 | 0.121 | 0.118 | 0.187 | 0.889 | 0.708 |
| Race.PC10 | 0.0913 | 0.237 | 0.314 | 0.869 | 0.179 | 0.482 | 0.0819 | 0.963 | 0.957 |

### Paternal Characteristics

Paternal performance on the adult SRS form ranged from 6 to 82. Fathers were predominantly White and non-Hispanic (80%), and their ages ranged between 28 to 51.2 years of age (Table 5). There were no significant associations of paternal age, smoking status, race, education, or paternity status with paternal SRS scores. Similarly, there were no significant associations of paternal age, smoking status, race, or paternity status with child SRS scores. There was, however, a significant association of paternal education with child SRS scores (p<0.05, Table 5).

TABLE 5
Bivariate associations of Phenotypic Assessment
Scores with Demographic & Laboratory Variables

| DAD.SRS | | | CHILD.SRS | | |
|---|---|---|---|---|---|
| (n = 45) | | | (N = 31) | | |
| | Mean | | | Mean | |
| N (%) | (SD) | P-value | N (%) | (SD) | P-value |

Paternal Factors

| | | | | | | |
|---|---|---|---|---|---|---|
| Dad SRS | 23 | 29.58 | — | 21 | 26.93 | 9.68E−01 |
| (Median, IQR) | (10.5, 35.5) | (19.63) | | (14, 28) | (18.92) | |
| Paternal Age | — | 36.96 | 3.88E−01 | — | 36.54 | 0.062805446 |
| (years) | | (5.66) | | | (6.22) | |
| Paternal Smoking | | | 3.57E−01 | | | 5.54E−01 |
| (ever) | | | | | | |
| Yes | 21 | 26.38 | | 12 | 36.83 | |
| | (46.7) | (18.78) | | (38.7) | (24.38) | |
| No | 19 | 31.58 | | 14 | 36.57 | |
| | (42.2) | (20.62) | | (45.2) | (33.57) | |
| Missing | 5 | 35.40 | | 5 | 54 | |
| | (10.4) | (21.13) | | (16.1) | (22.59) | |
| Paternal Race, | | | 8.59E−01 | | | 0.197548634 |
| Ethnicity | | | | | | |
| White | 36 | 30.83 | | 25 | 41.48 | |
| | (80) | (20.66) | | (80.6) | (30.78) | |
| Black | 4 | 23.5 | | 2 | 20.5 | |
| | (8.9) | (19.97) | | (6.5) | (6; 36) | |
| Asian | 2 | 28.5 | | 1 | 16 | |
| | (4.4) | (14.85) | | (3.2) | (NA) | |
| Other | 3 | 23.33 | | 3 | 43.33 | |
| | (6.7) | (11.59) | | (9.7) | (12.06) | |
| Paternal Education | | | 2.34E−01 | | | 0.024340811 |
| Less than HS | — | — | | — | — | |
| HS Diploma/GED | 7 | 36.00 | | 5 | 62 | |
| | (14.6) | (20.18) | | (16.1) | (26.98) | |
| Some college | 7 | 32.86 | | 8 | 50.88 | |
| | (14.6) | (23.34) | | (25.8) | (39.44) | |
| Bachelor's | 12 | 24.75 | | 7 | 23.29 | |
| degree | (25.0) | (17.89) | | (22.6) | (10.16) | |
| Graduate/ | 15 | 24.93 | | 11 | 31.27 | |
| Professional | (31.2) | (18.31) | | (35.5) | (19.78) | |
| Degree | | | | | | |
| Missing | 7 | 35.43 | | — | — | |
| | (14.6) | (20.41) | | | | |
| Paternity status | | | 3.63E−01 | | | 0.94016653 |
| Proband & sibling | 38 | 26.61 | | 25 | 36.92 | |
| | (84.4) | (19.31) | | (80.6) | (29.34) | |
| Sibling only | 7 | 34.86 | | 6 | 37.67 | |
| | (15.6) | (22.06) | | (19.4) | (27.56) | |

Child Factors

| | | | | | | |
|---|---|---|---|---|---|---|
| Child SRS | 28 | 37.90 | 9.68E−01 | 29 | 39.48 | — |
| (Median, IQR) | (16, 40) | (28.35) | | (14.25, 43.75) | (28.57) | |
| Child AOSI | 4 | 5.4 | 2.00E−02 | 5 | 6.23 | 0.201906388 |
| 12 months | (1, 7) | (4.15) | | (2.5, 7.5) | (4.61) | |
| (Median, IQR) | | | | | | |
| Gestational Age | — | 39.35 | 8.19E−01 | — | 39.49 | 0.073061619 |
| (weeks) | | (1.64) | | | (1.58) | |
| Birthweight | — | 3436.36 | 3.53E−01 | — | 3524.46 | 0.903943003 |
| (grams) | | (662.03) | | | (534.14) | |
| Offspring Sex | | | 7.30E−01 | | | 0.001750471 |
| Female | 19 | 27.89 | | 10 | 21 | |
| | (42.2) | (17.4) | | (32.3) | (9.9) | |
| Male | 26 | 30.81 | | 21 | 48.29 | |
| | (57.8) | (21.36) | | (67.7) | (30.46) | |
| Study site | | | 7.47E−01 | | | 0.621530934 |
| Drexel | 14 | 29.71 | | 8 | 33.25 | |
| | (29.71) | (17.98) | | (25.8) | (26.18) | |
| Johns Hopkins | 16 | 30.94 | | 10 | 51.1 | |
| | (35.6) | (25.48) | | (32.3) | (38.09) | |
| Kaiser | 7 | 33.29 | | 6 | 37.67 | |
| | (15.6) | (18.38) | | (19.4) | (25.66) | |
| UC Davis | 8 | 23.38 | | 7 | 31.57 | |
| | (17.8) | (8.91) | | (22.6) | (14.23) | |

Laboratory Factors

| | | | | | | |
|---|---|---|---|---|---|---|
| Hybridization date | — | — | 6.66E−01 | — | — | 3.78E−01 |
| Shearing date | — | — | 1.55E−01 | — | — | 0.433214574 |
| Shearing Matching | — | — | 4.36E−01 | — | — | 0.88525823 |
| CHARM Gel | — | — | 3.49E−01 | — | — | 4.04E−01 |
| Gel location | — | — | 1.87E−01 | — | — | 3.08E−01 |

*For continuous variables, Spearman correlation tests were performed. For dichotomous variables, a Mann-Whitney/Wilcoxon rank-sum test was performed. For nominal variables a Kruskal-Wallis test was performed. Numbers reported in the first column are the number and corresponding percent of the analytic population unless otherwise specified. For the “ paternity status” variable, “proband and sibling” indicates that the father was a biological father to both the older sibling already diagnosed with ASD (the EARLI proband), and the younger sibling that was the focus of EARLI, while “sibling only” indicates that the father was a biological father to only the younger sibling.

### Child Characteristics

Child performance on the SRS form ranged from 6-133. No significant associations were found between gestational age, birthweight, BSRC group, offspring sex, or study enrollment site and paternal SRS scores. There were similarly no signification associations between offspring AOSI at 12 months, gestational age, birthweight, or study enrollment site and child SRS scores. We did identify a significant relationship between offspring AOSI scores at 12 months and paternal SRS scores (p<0.05), as well as a significant relationship between child sex and child SRS scores (p<0.01).

### Relationship Between Paternal and Offspring SRS Scores

Given that there is evidence in the literature to support the heritability of autistic traits with respect to SRS (3), we asked whether paternal and child SRS scores were significantly associated with each other. We did not observe a significant relationship between paternal and child SRS (p=0.97).

### DNA Methylation and SRS Score Analyses

### DNA Methylation and Child SRS Score

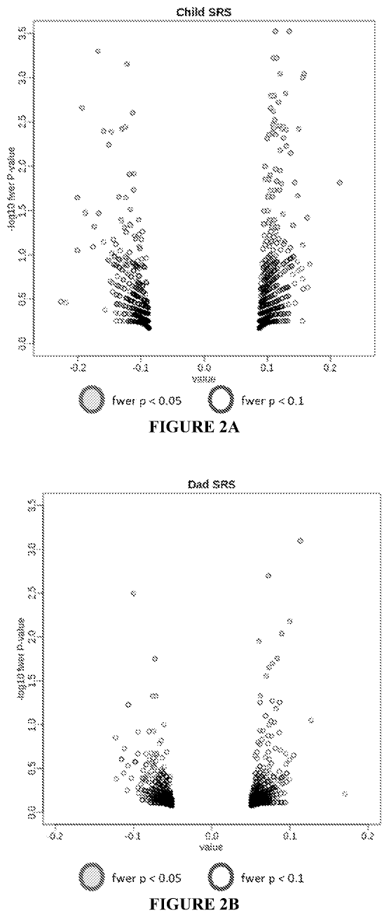

Given that we did not observe a heritable relationship between paternal and child SRS scores, we asked whether paternal epigenetic information might be associated with autistic traits in children in the EARLI cohort. Using a bump-hunting method we identified 1482 differentially methylated regions (DMRs) in sperm that were associated with child SRS scores at 36 months. After permutation analyses, 94 DMRs remained significant (fwer p<0.05, FIGS. 2A-B). Of the 94 significant DMRs associated with child SRS scores, the top 14 DMRs are shown in Table 6 (Table 7). Of interest, we see that the genes associated with those DMRs share common functions in synaptic function, neurogenesis, and development. To determine whether genes associated with all 94 significant DMRs had known roles in autism, we examined a list of curated by SFARI that support the gene's relevance to ASD risk. We found significant overlap between these two datasets, with 14 genes in common between the two (p<0.001, OR=3.25).

TABLE 6
Child DMRs (top 14) with functions and ASD-associations (known or new)

| | FWER | | Genic | | Known Association |
|---|---|---|---|---|---|
| Genomic Location | p value | Symbol | Location | Function | with ASD |
| chr16: 79027119- | 0 | WWOX | inside intron | Oxidoreductase involved in DNA | Previously known |
| 79030054 | | | | damage repair mechanisms and | association (SFARI |
| | | | | neuronal signaling | Category 2) |
| chr16: 7065694- | 0 | A2BP1 | inside intron | RNA binding protein involved in | Previously known |
| 7068381 | | | | neuronal signlaing | association (SFARI |
| | | | | | Category 2) |
| chr18: 76744751- | 0 | SALL3 | inside intron | Inhibits DNMT3A fuction at CpG | Previously known |
| 76746907 | | | | islands; roles in embryonic | association |
| | | | | development | |
| chr16: 78376735- | 0 | WWOX | inside intron | Oxidoreductase involved in DNA | Previously known |
| 78378445 | | | | damage repair mechanisms and | association (SFARI |
| | | | | neuronal signaling | Category 2) |
| chr10: 1894909- | 0.00010004 | ADARB2 | upstream | Involved in synaptic function | Previously known |
| 1896546 | | | | | association |
| chr16: 78974715- | 0.00010004 | WWOX | inside intron | Oxidoreductase involved in DNA | Previously known |
| 78976580 | | | | damage repair mechanisms and | association (SFARI |
| | | | | neuronal signaling | Category 2) |
| chr10: 3057233- | 0.00030012 | PFKP | upstream | Involved in regulating glycolysis | Previously known |
| 3058630 | | | | | association |
| chr19: 30745777- | 0.00030012 | ZNF536 | upstream | involved in synaptic function and | novel association |
| 30747506 | | | | negative regulation of neuronal | |
| | | | | differentiation | |
| chr4: 177804604- | 0.0005002 | VEGFC | upstream | Propmotes angiogenesis and | Previously known |
| 177805652 | | | | endothelial cell growth | association |
| chr18: 75687429- | 0.00060024 | GALR1 | downstream | Receptor for the galanin neuropeptide; | Previously known |
| 75689009 | | | | inhibits adenylyl cyclase via the | association |
| | | | | Gi/Go G protein family | |
| chr16: 6334658- | 0.00060024 | A2BP1 | inside intron | RNA binding protein involved in | Previously known |
| 6336272 | | | | neuronal signlaing | association (SFARI |
| | | | | | Category 2) |
| chr11: 133989860- | 0.00070028 | JAM3 | inside intron | Junctional adhesion protein involved | novel association |
| 133991280 | | | | in homin and mobilization of | |
| | | | | hematopoietic stem and progenitors | |
| | | | | within bone marrow; plays a role in | |
| | | | | spermatogenesis | |
| chr14: 99254155- | 0.00090036 | C14orf177 | downstream | limited literature | novel association |
| 99255193 | | | | | |
| chrX: 103301728- | 0.00090036 | H2BFM | downstream | Core component of nucleosomes; | novel association |
| 103303214 | | | | regulates DNA accessibility | |

The top 14 significant DMRs in paternal sperm associated with SRS scores in 36-month-old children. The boundaries of the DMR are shown in the genomic location column; the fwer p value is displayed alongside the gene symbol. The genic location characterized where within the gene body the DMR is located. Gene functions were taken from the human protein atlas (proteinatlas.org) as well as gene cards (genecards.org). Associations with ASD were determined by literature describing associations of the gene with autism. SFARI category is defined by the Simons Foundation for Autism Research Initiative (SFARI) based on their scoring algorithm. A score of 2 reflects a strong candidate. Genes that did not meet this criteria are termed “novel association”.

TABLE 7
All Child DMRs

| chr | start | end | value | area | pns | indexStart | indexEnd |
|---|---|---|---|---|---|---|---|
| chr16 | 79027119 | 79030054 | 0.159216512 | 6.527877006 | 93556 | 1392972 | 1393012 |
| chr16 | 7065694 | 7068381 | 0.139654055 | 5.446508137 | 86677 | 1298460 | 1298498 |
| chr18 | 76744751 | 76746907 | 0.14172677 | 4.535256646 | 115312 | 1724753 | 1724784 |
| chr16 | 78376735 | 78378445 | 0.159570647 | 3.989266182 | 93415 | 1390667 | 1390691 |
| chr10 | 1894909 | 1896546 | 0.137069427 | 3.289666238 | 22038 | 328194 | 328217 |
| chr16 | 78974715 | 78976580 | 0.113359657 | 3.060710744 | 93543 | 1392691 | 1392717 |
| chr10 | 3057233 | 3058630 | 0.134737338 | 2.82948409 | 22271 | 332096 | 332116 |
| chr19 | 30745777 | 30747506 | 0.11293205 | 2.823301239 | 120859 | 1798459 | 1798483 |
| chr4 | 177804604 | 177805652 | −0.1683934 | 2.694294437 | 187727 | 2777709 | 2777724 |
| chr18 | 75687429 | 75689009 | 0.114647835 | 2.636900211 | 115020 | 1718137 | 1718159 |
| chr16 | 6334658 | 6336272 | 0.109021922 | 2.616526134 | 86562 | 1296961 | 1296984 |
| chr11 | 133989860 | 133991280 | −0.12222157 | 2.566652998 | 48936 | 744222 | 744242 |
| chr14 | 99254155 | 99255193 | 0.158108255 | 2.529732088 | 73934 | 1105299 | 1105314 |
| chrX | 103301728 | 103303214 | 0.120043492 | 2.52091333 | 256267 | 3773583 | 3773603 |
| chr5 | 81183133 | 81184172 | 0.156167824 | 2.498685184 | 194870 | 2883503 | 2883518 |
| chr16 | 79006043 | 79007310 | 0.128831082 | 2.447790558 | 93548 | 1392813 | 1392831 |
| chr11 | 133523204 | 133524674 | 0.110685063 | 2.435071391 | 48833 | 742112 | 742133 |
| chr6 | 169110451 | 169111980 | 0.105579936 | 2.428338526 | 214642 | 3167550 | 3167572 |
| chrX | 68382157 | 68383487 | 0.117626624 | 2.352532485 | 255488 | 3762693 | 3762712 |
| chr6 | 3082883 | 3083659 | −0.19360309 | 2.323237107 | 202731 | 2994488 | 2994499 |
| chr10 | 16932229 | 16933742 | 0.104884956 | 2.307469036 | 24538 | 366593 | 366614 |
| chr8 | 70380052 | 70381614 | 0.109166796 | 2.29250271 | 235616 | 3479256 | 3479276 |
| chr4 | 89680075 | 89681417 | −0.11313175 | 2.262634921 | 184620 | 2738288 | 2738307 |
| chr12 | 129731581 | 129732908 | 0.111785273 | 2.235705454 | 59562 | 896205 | 896224 |
| chr8 | 3564640 | 3565970 | 0.1082423 | 2.164845991 | 229614 | 3392880 | 3392899 |
| chr7 | 158018644 | 158019928 | 0.113425562 | 2.155085678 | 228421 | 3370458 | 3370476 |
| chr8 | 140527497 | 140528747 | 0.113259192 | 2.15192465 | 239390 | 3527075 | 3527093 |
| chr6 | 166959009 | 166960197 | 0.118802355 | 2.138442396 | 214081 | 3156542 | 3156559 |
| chr5 | 2632787 | 2633963 | 0.125196194 | 2.128335299 | 190126 | 2815432 | 2815448 |
| chr18 | 3747209 | 3748310 | −0.12466754 | 2.119348215 | 108864 | 1627460 | 1627476 |
| chr15 | 86604965 | 86606253 | 0.110271802 | 2.095164229 | 82484 | 1235513 | 1235531 |
| chr7 | 157991932 | 157992826 | 0.149553239 | 2.093745351 | 228417 | 3370420 | 3370433 |
| chr10 | 43731010 | 43732120 | 0.122905522 | 2.089393869 | 26564 | 394613 | 394629 |
| chr10 | 13868186 | 13869238 | −0.13008868 | 2.081418954 | 24120 | 360800 | 360815 |
| chr4 | 140925039 | 140926209 | 0.129797371 | 2.076757941 | 186120 | 2757024 | 2757039 |
| chr7 | 150217031 | 150217910 | −0.15894282 | 2.066256673 | 226795 | 3340990 | 3341002 |
| chr6 | 10824173 | 10825091 | −0.14711599 | 2.059623881 | 203800 | 3011177 | 3011190 |
| chr10 | 127245100 | 127246369 | 0.108102267 | 2.053943081 | 34274 | 510130 | 510148 |
| chr18 | 76064588 | 76066099 | 0.102419782 | 2.048395639 | 115105 | 1720011 | 1720030 |
| chr17 | 70261462 | 70262684 | 0.113632571 | 2.045386283 | 105475 | 1572632 | 1572649 |
| chr6 | 168549886 | 168550920 | 0.125391664 | 2.006266624 | 214467 | 3163859 | 3163874 |
| chr16 | 54574022 | 54575370 | 0.099764477 | 1.995289544 | 90763 | 1352947 | 1352966 |
| chr6 | 1124560 | 1125411 | −0.15082515 | 1.960726935 | 202349 | 2987863 | 2987875 |
| chr6 | 164373291 | 164374257 | 0.129947211 | 1.949208166 | 213686 | 3149550 | 3149564 |
| chr3 | 3079927 | 3080979 | 0.120509752 | 1.928156025 | 165662 | 2473326 | 2473341 |
| chr16 | 6695982 | 6696898 | 0.136996557 | 1.917951796 | 86628 | 1297801 | 1297814 |
| chr10 | 130758409 | 130759674 | 0.0963894 | 1.831398608 | 35000 | 523957 | 523975 |
| chr1 | 247710605 | 247711703 | 0.113096494 | 1.809543907 | 21464 | 316611 | 316626 |
| chr6 | 169086098 | 169087090 | 0.120146804 | 1.802202054 | 214636 | 3167385 | 3167399 |
| chr6 | 148467734 | 148468789 | −0.11126147 | 1.780183546 | 211432 | 3115897 | 3115912 |
| chr3 | 39309180 | 39310152 | −0.11855764 | 1.778364543 | 168557 | 2513572 | 2513586 |
| chr18 | 75832003 | 75833196 | 0.104209148 | 1.77155552 | 115050 | 1718638 | 1718654 |
| chr8 | 2793232 | 2794464 | 0.096661479 | 1.739906615 | 229440 | 3390235 | 3390252 |
| chr4 | 182772268 | 182773421 | 0.102144996 | 1.736464931 | 187999 | 2781085 | 2781101 |
| chr1 | 5035583 | 5036631 | 0.114973339 | 1.724600091 | 1279 | 22185 | 22199 |
| chr4 | 55807825 | 55808591 | 0.143269852 | 1.71923823 | 183486 | 2724421 | 2724432 |
| chr10 | 123348606 | 123349109 | 0.214700518 | 1.717604145 | 33450 | 495695 | 495702 |
| chr8 | 6084773 | 6085752 | 0.121928845 | 1.707003835 | 230015 | 3398828 | 3398841 |
| chr11 | 134013860 | 134014820 | −0.11170914 | 1.675637087 | 48942 | 744313 | 744327 |
| chr12 | 129762460 | 129763502 | 0.104337574 | 1.669401177 | 59567 | 896289 | 896304 |
| chr7 | 158090996 | 158091922 | 0.119160252 | 1.668243523 | 228445 | 3370951 | 3370964 |
| chr1 | 214636479 | 214637459 | 0.110885201 | 1.663278009 | 17974 | 265435 | 265449 |
| chr7 | 158034305 | 158034995 | 0.148033244 | 1.628365686 | 228425 | 3370527 | 3370537 |
| chr1 | 240070784 | 240071832 | 0.101556984 | 1.624911746 | 20597 | 303766 | 303781 |
| chr10 | 130622921 | 130623905 | 0.108122991 | 1.621844865 | 34969 | 523186 | 523200 |
| chr2 | 60030337 | 60031119 | −0.13501578 | 1.62018932 | 132235 | 1968817 | 1968828 |
| chr8 | 136364091 | 136365129 | 0.101158256 | 1.618532102 | 239045 | 3522613 | 3522628 |
| chr6 | 33830454 | 33831276 | −0.12399329 | 1.611912723 | 205624 | 3037690 | 3037702 |
| chr4 | 79428290 | 79428774 | −0.20096666 | 1.607733315 | 184163 | 2732637 | 2732644 |
| chr11 | 134015498 | 134016338 | −0.11719019 | 1.523472426 | 48943 | 744328 | 744340 |
| chr8 | 130257804 | 130258893 | 0.101129344 | 1.516940167 | 238474 | 3515269 | 3515283 |
| chr14 | 93705210 | 93706287 | 0.094616789 | 1.513868616 | 73032 | 1091104 | 1091119 |
| chr8 | 77867266 | 77867740 | −0.18864544 | 1.509163505 | 236028 | 3484786 | 3484793 |
| chr12 | 29815765 | 29816325 | −0.16698988 | 1.502908891 | 51865 | 789976 | 789984 |
| chr9 | 75415158 | 75415654 | −0.18785515 | 1.502841231 | 243622 | 3589231 | 3589238 |
| chr8 | 136281153 | 136281905 | 0.124872591 | 1.498471092 | 239039 | 3522543 | 3522554 |
| chr9 | 106081923 | 106082489 | 0.162896632 | 1.466069684 | 246363 | 3629413 | 3629421 |
| chr12 | 79197120 | 79198158 | −0.13191134 | 1.451024717 | 54671 | 831284 | 831294 |
| chr16 | 8112733 | 8113628 | 0.103622078 | 1.450709093 | 86829 | 1300790 | 1300803 |
| chr3 | 58671808 | 58672720 | −0.10358979 | 1.450257032 | 170838 | 2549186 | 2549199 |
| chr6 | 169091834 | 169092666 | 0.111551508 | 1.450169606 | 214637 | 3167409 | 3167421 |
| chr19 | 55529476 | 55530178 | −0.1311895 | 1.443084488 | 125161 | 1860958 | 1860968 |
| chr10 | 3282005 | 3283002 | 0.101977992 | 1.427691882 | 22347 | 333371 | 333384 |
| chr2 | 21229876 | 21230848 | 0.094524345 | 1.417865173 | 129006 | 1922518 | 1922532 |
| chr8 | 136247572 | 136248412 | 0.108968192 | 1.416586491 | 239038 | 3522530 | 3522542 |
| chr4 | 182786796 | 182787776 | 0.094387538 | 1.415813074 | 188002 | 2781164 | 2781178 |
| chr1 | 223159390 | 223160144 | −0.11755479 | 1.41065751 | 18440 | 271499 | 271510 |
| chr5 | 34491468 | 34492114 | 0.140548715 | 1.405487152 | 192713 | 2854760 | 2854769 |
| chr10 | 75863586 | 75864359 | −0.11697221 | 1.403666573 | 28793 | 426953 | 426964 |
| chr6 | 25319656 | 25320232 | −0.17422975 | 1.393838007 | 205212 | 3031049 | 3031056 |
| chr17 | 70388426 | 70389286 | 0.107077288 | 1.392004738 | 105501 | 1573098 | 1573110 |
| chr8 | 3224324 | 3225118 | 0.11589469 | 1.390736281 | 229549 | 3391872 | 3391883 |
| chr10 | 366778 | 367458 | −0.12610404 | 1.387144443 | 21600 | 318594 | 318604 |
| chr16 | 78776493 | 78777317 | 0.106614555 | 1.385989221 | 93499 | 1392062 | 1392074 |

| | | pval_fwer_{—} | | | | |
|---|---|---|---|---|---|---|
| chr | L | max | pval_pool | qval_pool | name | annotation |
| chr16 | 41 | 0 | 3.86E−05 | 0.008283866 | WWOX | NM_016373 |
| chr16 | 39 | 0 | 3.86E−05 | 0.008283866 | A2BP1 | NM_001142334 |
| chr18 | 32 | 0 | 3.86E−05 | 0.008283866 | SALL3 | NM_171999 |
| chr16 | 25 | 0 | 3.86E−05 | 0.008283866 | WWOX | NM_016373 |
| chr10 | 24 | 0.00010004 | 3.86E−05 | 0.008283866 | ADARB2 | NM_018702 |
| chr16 | 27 | 0.00010004 | 3.86E−05 | 0.008283866 | WWOX | NM_016373 |
| chr10 | 21 | 0.00030012 | 0.000115888 | 0.018638699 | PFKP | NM_002627 |
| chr19 | 25 | 0.00030012 | 0.000115888 | 0.018638699 | ZNF536 | NM_014717 |
| chr4 | 16 | 0.0005002 | 0.000193147 | 0.027110835 | VEGFC | NM_005429 |
| chr18 | 23 | 0.00060024 | 0.000231777 | 0.027110835 | GALR1 | NM_001480 |
| chr16 | 24 | 0.00060024 | 0.000231777 | 0.027110835 | A2BP1 | NM_018723 |
| chr11 | 21 | 0.00070028 | 0.000270406 | 0.028993532 | JAM3 | NM_032801 |
| chr14 | 16 | 0.00090036 | 0.000347665 | 0.031952055 | C14orf177 | NM_182560 |
| chrX | 21 | 0.00090036 | 0.000347665 | 0.031952055 | H2BFM | NM_001164416 |
| chr5 | 16 | 0.0010004 | 0.000386294 | 0.033135465 | ATG10 | NM_001131028 |
| chr16 | 19 | 0.0015006 | 0.000579441 | 0.04418062 | WWOX | NM_016373 |
| chr11 | 22 | 0.00160064 | 0.000618071 | 0.04418062 | OPCML | NM_001012393 |
| chr6 | 23 | 0.00160064 | 0.000618071 | 0.04418062 | SMOC2 | NM_001166412 |
| chrX | 20 | 0.00190076 | 0.000733959 | 0.049703197 | PJA1 | NM_001032396 |
| chr6 | 12 | 0.00220088 | 0.000849847 | 0.052070016 | RIPK1 | NM_003804 |
| chr10 | 22 | 0.00220088 | 0.000849847 | 0.052070016 | CUBN | NM_001081 |
| chr8 | 21 | 0.00240096 | 0.000927106 | 0.054025214 | SULF1 | NM_015170 |
| chr4 | 20 | 0.002501 | 0.000965736 | 0.054025214 | FAM13A | NM_001015045 |
| chr12 | 20 | 0.0030012 | 0.001158883 | 0.054673517 | TMEM132D | NM_133448 |
| chr8 | 20 | 0.003401361 | 0.001313401 | 0.054673517 | CSMD1 | NM_033225 |
| chr7 | 19 | 0.003501401 | 0.00135203 | 0.054673517 | PTPRN2 | NM_002847 |
| chr8 | 19 | 0.003501401 | 0.00135203 | 0.054673517 | KCNK9 | NM_016601 |
| chr6 | 18 | 0.003501401 | 0.00135203 | 0.054673517 | RPS6KA2 | NM_021135 |
| chr5 | 17 | 0.003601441 | 0.001390659 | 0.054673517 | IRX2 | NM_001134222 |
| chr18 | 17 | 0.003601441 | 0.001390659 | 0.054673517 | DLGAP1 | NM_001003809 |
| chr15 | 19 | 0.003701481 | 0.001429289 | 0.054673517 | AGBL1 | NM_152336 |
| chr7 | 14 | 0.003801521 | 0.001467918 | 0.054673517 | PTPRN2 | NM_002847 |
| chr10 | 17 | 0.003801521 | 0.001467918 | 0.054673517 | RASGEF1A | NM_145313 |
| chr10 | 16 | 0.003801521 | 0.001467918 | 0.054673517 | FRMD4A | NM_018027 |
| chr4 | 16 | 0.003901561 | 0.001506548 | 0.054673517 | MAML3 | NM_018717 |
| chr7 | 13 | 0.004001601 | 0.001545177 | 0.054673517 | GIMAP7 | NM_153236 |
| chr6 | 14 | 0.004101641 | 0.001583807 | 0.054673517 | MAK | NM_005906 |
| chr10 | 19 | 0.004201681 | 0.001622436 | 0.054673517 | LOC100169752 | NR_023362 |
| chr18 | 20 | 0.004401761 | 0.001699695 | 0.054673517 | SALL3 | NM_171999 |
| chr17 | 18 | 0.004401761 | 0.001699695 | 0.054673517 | SOX9 | NM_000346 |
| chr6 | 16 | 0.004801921 | 0.001892842 | 0.059170473 | FRMD1 | NM_024919 |
| chr16 | 20 | 0.004901961 | 0.001931471 | 0.059170473 | IRX3 | NM_024336 |
| chr6 | 13 | 0.005702281 | 0.002240507 | 0.067041522 | LOC285768 | NR_027115 |
| chr6 | 15 | 0.005902361 | 0.002317766 | 0.067777087 | QKI | NM_006775 |
| chr3 | 16 | 0.006602641 | 0.002588172 | 0.074002538 | CNTN4 | NM_175613 |
| chr16 | 14 | 0.007102841 | 0.002781319 | 0.077796309 | A2BP1 | NM_018723 |
| chr10 | 19 | 0.010004002 | 0.003978831 | 0.108924028 | MGMT | NM_002412 |
| chr1 | 16 | 0.010804322 | 0.004287866 | 0.114938644 | C1orf150 | NM_145278 |
| chr6 | 15 | 0.011204482 | 0.004442384 | 0.116650361 | SMOC2 | NM_001166412 |
| chr6 | 16 | 0.012204882 | 0.004867308 | 0.123770707 | SASH1 | NM_015278 |
| chr3 | 15 | 0.012304922 | 0.004905937 | 0.123770707 | CX3CR1 | NM_001337 |
| chr18 | 17 | 0.012605042 | 0.005060455 | 0.125213824 | GALR1 | NM_001480 |
| chr8 | 18 | 0.014105642 | 0.005678526 | 0.135303148 | CSMD1 | NM_033225 |
| chr4 | 17 | 0.014105642 | 0.005678526 | 0.135303148 | MGC45800 | NR_027107 |
| chr1 | 15 | 0.014705882 | 0.005910302 | 0.138265258 | AJAP1 | NM_018836 |
| chr4 | 12 | 0.015306122 | 0.006142079 | 0.139517747 | KDR | NM_002253 |
| chr10 | 8 | 0.015406162 | 0.006180708 | 0.139517747 | FGFR2 | NM_001144913 |
| chr8 | 14 | 0.015806323 | 0.006335226 | 0.140540075 | MCPH1 | NM_024596 |
| chr11 | 15 | 0.018607443 | 0.00745548 | 0.157126236 | JAM3 | NM_032801 |
| chr12 | 16 | 0.018707483 | 0.007494109 | 0.157126236 | TMEM132D | NM_133448 |
| chr7 | 14 | 0.018707483 | 0.007494109 | 0.157126236 | PTPRN2 | NM_002847 |
| chr1 | 15 | 0.018807523 | 0.007571368 | 0.157126236 | PTPN14 | NM_005401 |
| chr7 | 11 | 0.021508603 | 0.008652992 | 0.169279005 | PTPRN2 | NM_002847 |
| chr1 | 16 | 0.021708683 | 0.008730251 | 0.169279005 | CHRM3 | NM_000740 |
| chr10 | 15 | 0.022108844 | 0.008884768 | 0.169279005 | MGMT | NM_002412 |
| chr2 | 12 | 0.022208884 | 0.008923398 | 0.169279005 | BCL11A | NM_138559 |
| chr8 | 16 | 0.022308924 | 0.008962027 | 0.169279005 | LOC286094 | NR_026706 |
| chr6 | 13 | 0.022509004 | 0.009039286 | 0.169279005 | MLN | NM_002418 |
| chr4 | 8 | 0.022609044 | 0.009077916 | 0.169279005 | FRAS1 | NM_025074 |
| chr11 | 13 | 0.030712285 | 0.012477305 | 0.229344753 | JAM3 | NM_032801 |
| chr8 | 15 | 0.031712685 | 0.012863599 | 0.233114995 | GSDMC | NM_031415 |
| chr14 | 16 | 0.032312925 | 0.013095376 | 0.23401922 | BTBD7 | NM_001002860 |
| chr8 | 8 | 0.033113245 | 0.013404411 | 0.236260403 | PXMP3 | NM_000318 |
| chr12 | 9 | 0.033913565 | 0.013790706 | 0.236587219 | TMTC1 | NM_175861 |
| chr9 | 8 | 0.033913565 | 0.013790706 | 0.236587219 | TMC1 | NM_138691 |
| chr8 | 12 | 0.034713886 | 0.014099741 | 0.238706145 | LOC286094 | NR_026706 |
| chr9 | 9 | 0.038215286 | 0.015644918 | 0.261425908 | CYLC2 | NM_001340 |
| chr12 | 11 | 0.040216086 | 0.016456136 | 0.26201562 | SYT1 | NM_001135805 |
| chr16 | 14 | 0.040316126 | 0.016494766 | 0.26201562 | A2BP1 | NM_145893 |
| chr3 | 14 | 0.040316126 | 0.016494766 | 0.26201562 | FAM3D | NM_138805 |
| chr6 | 13 | 0.040316126 | 0.016494766 | 0.26201562 | SMOC2 | NM_001166412 |
| chr19 | 11 | 0.041516607 | 0.016958319 | 0.266093946 | GP6 | NM_001083899 |
| chr10 | 14 | 0.043617447 | 0.017885425 | 0.277260004 | PITRM1 | NM_014889 |
| chr2 | 15 | 0.044617847 | 0.018464867 | 0.277991138 | APOB | NM_000384 |
| chr8 | 13 | 0.044917967 | 0.018580755 | 0.277991138 | LOC286094 | NR_026706 |
| chr4 | 15 | 0.044917967 | 0.018580755 | 0.277991138 | MGC45800 | NR_027107 |
| chr1 | 12 | 0.045618247 | 0.01892842 | 0.279789908 | DISP1 | NM_032890 |
| chr5 | 10 | 0.046518607 | 0.019314714 | 0.279789908 | RAI14 | NM_001145522 |
| chr10 | 12 | 0.046618647 | 0.019353343 | 0.279789908 | VCL | NM_003373 |
| chr6 | 8 | 0.048219288 | 0.020010044 | 0.281298946 | LRRC16A | NM_017640 |
| chr17 | 13 | 0.048719488 | 0.020203191 | 0.281298946 | SLC39A11 | NM_139177 |
| chr8 | 12 | 0.048719488 | 0.02024182 | 0.281298946 | CSMD1 | NM_033225 |
| chr10 | 11 | 0.049219688 | 0.020434967 | 0.281298946 | DIP2C | NM_014974 |
| chr16 | 13 | 0.049519808 | 0.020550856 | 0.281298946 | WWOX | NM_016373 |

| | | | | | inside- | |
|---|---|---|---|---|---|---|
| chr | description | region | distance | subregion | distance | exonnumber |
| chr16 | inside | inside | 893569 | inside | 215450 | 9 |
| | intron | | | intron | | |
| chr16 | inside | inside | 241885 | inside | 33676 | 2 |
| | intron | | | intron | | |
| chr18 | inside | inside | 4477 | inside | −4396 | 1 |
| | intron | | | intron | | |
| chr16 | inside | inside | 243185 | inside | 42311 | 6 |
| | intron | | | intron | | |
| chr10 | upstream | upstream | 115192 | NA | NA | NA |
| chr16 | inside | inside | 841165 | inside | 268924 | 9 |
| | intron | | | intron | | |
| chr10 | upstream | upstream | 51121 | NA | NA | NA |
| chr19 | upstream | upstream | 115821 | NA | NA | NA |
| chr4 | upstream | upstream | 90710 | NA | NA | NA |
| chr18 | downstream | downstream | 725422 | NA | NA | NA |
| chr16 | inside | inside | 265527 | inside | 30723 | 2 |
| | intron | | | intron | | |
| chr11 | inside | inside | 51041 | inside | 18465 | 2 |
| | intron | | | intron | | |
| chr14 | downstream | downstream | 76206 | NA | NA | NA |
| chrX | downstream | downstream | 7213 | NA | NA | NA |
| chr5 | upstream | upstream | 83671 | NA | NA | NA |
| chr16 | inside | inside | 872493 | inside | 238194 | 9 |
| | intron | | | intron | | |
| chr11 | upstream | upstream | 120802 | NA | NA | NA |
| chr6 | downstream | downstream | 268621 | NA | NA | NA |
| chrX | overlaps | inside | 1784 | overlaps | 0 | 2 |
| | exon | | | exon | | |
| | upstream | | | upstream | | |
| chr6 | covers | inside | 5826 | covers | 0 | 4 |
| | exon(s) | | | exon(s) | | |
| chr10 | covers | inside | 238073 | covers | 0 | 55 |
| | exon(s) | | | exon(s) | | |
| chr8 | inside | inside | 1194 | inside | −868 | 1 |
| | intron | | | intron | | |
| chr4 | inside | inside | 62984 | inside | 24 | 8 |
| | intron | | | intron | | |
| chr12 | inside | inside | 655303 | inside | 37374 | 5 |
| | intron | | | intron | | |
| chr8 | overlaps | inside | 1286357 | overlaps | 0 | 7 |
| | exon | | | exon | | |
| | downstream | | | downstream | | |
| chr7 | inside | inside | 360553 | inside | 20680 | 4 |
| | intron | | | intron | | |
| chr8 | downstream | downstream | 186551 | NA | NA | NA |
| chr6 | inside | inside | 80528 | inside | 6738 | 2 |
| | intron | | | intron | | |
| chr5 | downstream | downstream | 117805 | NA | NA | NA |
| chr18 | inside | inside | 96985 | inside | 4698 | 3 |
| | intron | | | intron | | |
| chr15 | upstream | upstream | 78988 | NA | NA | NA |
| chr7 | inside | inside | 387655 | inside | −5036 | 4 |
| | intron | | | intron | | |
| chr10 | inside | inside | 30246 | inside | 29441 | 2 |
| | intron | | | intron | | |
| chr10 | inside | inside | 503627 | inside | 15279 | 4 |
| | intron | | | intron | | |
| chr4 | inside | inside | 149023 | inside | 112919 | 2 |
| | intron | | | intron | | |
| chr7 | inside | inside | 5087 | inside | 0 | 2 |
| | exon | | | exon | | |
| chr6 | inside | inside | 6018 | inside | 5001 | 2 |
| | intron | | | intron | | |
| chr10 | upstream | upstream | 16570 | NA | NA | NA |
| chr18 | upstream | upstream | 674175 | NA | NA | NA |
| chr17 | downstream | downstream | 144302 | NA | NA | NA |
| chr6 | upstream | upstream | 70048 | NA | NA | NA |
| chr16 | upstream | upstream | 253645 | NA | NA | NA |
| chr6 | upstream | upstream | 22994 | NA | NA | NA |
| chr6 | downstream | downstream | 537617 | NA | NA | NA |
| chr3 | covers | inside | 146007 | covers | 0 | 10 |
| | exon(s) | | | exon(s) | | |
| chr16 | inside | inside | 626851 | inside | 7705 | 3 |
| | intron | | | intron | | |
| chr10 | upstream | upstream | 505779 | NA | NA | NA |
| chr1 | promoter | promoter | 747 | NA | NA | NA |
| chr6 | downstream | downstream | 244268 | NA | NA | NA |
| chr6 | upstream | upstream | 194939 | NA | NA | NA |
| chr3 | inside | inside | 11374 | inside | 1172 | 2 |
| | intron | | | intron | | |
| chr18 | downstream | downstream | 869996 | NA | NA | NA |
| chr8 | inside exon | inside | 2057863 | inside | 0 | 70 |
| | | | | exon | | |
| chr4 | downstream | downstream | 292246 | NA | NA | NA |
| chr1 | downstream | downstream | 320479 | NA | NA | NA |
| chr4 | downstream | downstream | 183170 | NA | NA | NA |
| chr10 | inside | inside | 4371 | inside | −4113 | 1 |
| | intron | | | intron | | |
| chr8 | upstream | upstream | 178368 | NA | NA | NA |
| chr11 | covers | inside | 75041 | covers | 0 | 5 |
| | exon(s) | | | exon(s) | | |
| chr12 | inside | inside | 624709 | inside | −58676 | 4 |
| | intron | | | intron | | |
| chr7 | inside | inside | 288559 | inside | −17588 | 3 |
| | intron | | | intron | | |
| chr1 | inside | inside | 87182 | inside | −513 | 2 |
| | intron | | | intron | | |
| chr7 | inside | inside | 345486 | inside | 36341 | 4 |
| | intron | | | intron | | |
| chr1 | inside | inside | 278412 | inside | 0 | 5 |
| | exon | | | exon | | |
| chr10 | upstream | upstream | 641548 | NA | NA | NA |
| chr2 | downstream | downstream | 749513 | NA | NA | NA |
| chr8 | downstream | downstream | 117718 | NA | NA | NA |
| chr6 | upstream | upstream | 58662 | NA | NA | NA |
| chr4 | covers | inside | 449567 | covers | 0 | 62 |
| | exon(s) | | | exon(s) | | |
| chr11 | covers | inside | 76679 | covers | 0 | 6 |
| | exon(s) | | | exon(s) | | |
| chr8 | downstream | downstream | 540240 | NA | NA | NA |
| chr14 | inside exon | inside | 93097 | inside | 0 | 11 |
| | | | | exon | | |
| chr8 | downstream | downstream | 44783 | NA | NA | NA |
| chr12 | inside | inside | 121366 | inside | 29497 | 6 |
| | intron | | | intron | | |
| chr9 | inside | inside | 278442 | inside | 4643 | 18 |
| | intron | | | intron | | |
| chr8 | inside | inside | 34780 | inside | 21508 | 3 |
| | intron | | | intron | | |
| chr9 | downstream | downstream | 324331 | NA | NA | NA |
| chr12 | upstream | upstream | 59614 | NA | NA | NA |
| chr16 | downstream | downstream | 729983 | NA | NA | NA |
| chr3 | upstream | upstream | 19248 | NA | NA | NA |
| chr6 | downstream | downstream | 250004 | NA | NA | NA |
| chr19 | covers | inside | 19453 | covers | 0 | 6 |
| | exon(s) | | | exon(s) | | |
| chr10 | upstream | upstream | 67003 | NA | NA | NA |
| chr2 | inside | inside | 36096 | inside | 0 | 26 |
| | exon | | | exon | | |
| chr8 | inside | inside | 1199 | inside | −814 | 1 |
| | intron | | | intron | | |
| chr4 | downstream | downstream | 277891 | NA | NA | NA |
| chr1 | inside | inside | 57608 | inside | −2940 | 3 |
| | intron | | | intron | | |
| chr5 | upstream | upstream | 164318 | NA | NA | NA |
| chr10 | overlaps | inside | 105715 | overlaps | 0 | 15 |
| | exon | | | exon downstream | | |
| | downstream | | | | | |
| chr6 | inside | inside | 40009 | inside | −34520 | 2 |
| | intron | | | intron | | |
| chr17 | downstream | downstream | 699566 | NA | NA | NA |
| chr8 | covers | inside | 1627209 | covers | 0 | 20 |
| | exon(s) | | | exon(s) | | |
| chr10 | inside | inside | 368149 | inside | −5487 | 31 |
| | intron | | | intron | | |
| chr16 | inside | inside | 642943 | inside | −309845 | 8 |
| | intron | | | intron | | |

| chr | nexons | UTR | strand | geneL | codingL | Rank |
|---|---|---|---|---|---|---|
| chr16 | 9 | inside | + | 1113012 | 1112017 | 1 |
| | | transcription | | | | |
| | | region | | | | |
| chr16 | 14 | 5′ UTR | + | 939530 | 658674 | 2 |
| chr18 | 3 | inside | + | 17916 | 17047 | 3 |
| | | transcription | | | | |
| | | region | | | | |
| chr16 | 9 | inside | + | 1113012 | 1112017 | 4 |
| | | transcription | | | | |
| | | region | | | | |
| chr10 | 10 | NA | − | 551644 | 550211 | 5 |
| chr16 | 9 | inside | + | 1113012 | 1112017 | 6 |
| | | transcription | | | | |
| | | region | | | | |
| chr10 | 22 | NA | + | 69243 | 68983 | 7 |
| chr19 | 5 | NA | + | 185637 | 113601 | 8 |
| chr4 | 7 | NA | − | 109204 | 108388 | 9 |
| chr18 | 3 | NA | + | 20086 | 18353 | 10 |
| chr16 | 16 | 5′ UTR | + | 1694208 | 658674 | 11 |
| chr11 | 9 | inside | + | 82829 | 80232 | 12 |
| | | transcription | | | | |
| | | region | | | | |
| chr14 | 4 | NA | + | 6147 | 1082 | 13 |
| chrX | 3 | NA | + | 2504 | 887 | 14 |
| chr5 | 9 | NA | + | 283367 | 265854 | 15 |
| chr16 | 9 | inside | + | 1113012 | 1112017 | 16 |
| | | transcription | | | | |
| | | region | | | | |
| chr11 | 8 | NA | − | 1117526 | 1112132 | 17 |
| chr6 | 13 | NA | + | 226842 | 225067 | 18 |
| chrX | 2 | overlaps | − | 4567 | 1766 | 19 |
| | | 5′ UTR | | | | |
| chr6 | 10 | inside | + | 38361 | 36515 | 20 |
| | | transcription | | | | |
| | | region | | | | |
| chr10 | 67 | inside | − | 305850 | 304790 | 21 |
| | | transcription | | | | |
| | | region | | | | |
| chr8 | 23 | 5′ UTR | + | 194288 | 94559 | 22 |
| chr4 | 18 | inside | − | 97296 | 94471 | 23 |
| | | transcription | | | | |
| | | region | | | | |
| chr12 | 9 | inside | − | 831941 | 829464 | 24 |
| | | transcription | | | | |
| | | region | | | | |
| chr8 | 70 | inside | − | 2059452 | 2055831 | 25 |
| | | transcription | | | | |
| | | region | | | | |
| chr7 | 23 | inside | − | 1048731 | 1046953 | 26 |
| | | transcription | | | | |
| | | region | | | | |
| chr8 | 3 | NA | − | 90495 | 84734 | 27 |
| chr6 | 21 | inside | − | 217872 | 214257 | 28 |
| | | transcription | | | | |
| | | region | | | | |
| chr5 | 5 | NA | − | 5488 | 3849 | 29 |
| chr18 | 10 | inside | − | 346459 | 346099 | 30 |
| | | transcription | | | | |
| | | region | | | | |
| chr15 | 24 | NA | + | 887041 | 885118 | 31 |
| chr7 | 23 | inside | − | 1048731 | 1046953 | 32 |
| | | transcription | | | | |
| | | region | | | | |
| chr10 | 13 | 5′ UTR | − | 72383 | 9872 | 33 |
| chr10 | 25 | inside | − | 687159 | 678196 | 34 |
| | | transcription | | | | |
| | | region | | | | |
| chr4 | 6 | inside | − | 437687 | 434004 | 35 |
| | | transcription | | | | |
| | | region | | | | |
| chr7 | 2 | overlaps | + | 6215 | 902 | 36 |
| | | 5′ UTR | | | | |
| chr6 | 13 | inside | − | 68153 | 66196 | 37 |
| | | transcription | | | | |
| | | region | | | | |
| chr10 | 5 | NA | + | 4072 | −1 | 38 |
| chr18 | 3 | NA | + | 17916 | 17047 | 39 |
| chr17 | 3 | NA | + | 5391 | 2995 | 40 |
| chr6 | 11 | NA | − | 23374 | 21997 | 41 |
| chr16 | 4 | NA | − | 3166 | 2364 | 42 |
| chr6 | 5 | NA | − | 140325 | −1 | 43 |
| chr6 | 8 | NA | + | 159217 | 155516 | 44 |
| chr3 | 16 | inside | + | 164118 | 155491 | 45 |
| | | transcription | | | | |
| | | region | | | | |
| chr16 | 16 | 5′ UTR | + | 1694208 | 658674 | 46 |
| chr10 | 5 | NA | + | 300329 | 299781 | 47 |
| chr1 | 5 | NA | + | 27395 | 25190 | 48 |
| chr6 | 13 | NA | + | 226842 | 225067 | 49 |
| chr6 | 20 | NA | + | 209455 | 205490 | 50 |
| chr3 | 2 | 5′ UTR | − | 16541 | 1067 | 51 |
| chr18 | 3 | NA | + | 20086 | 18353 | 52 |
| chr8 | 70 | 3′ UTR | − | 2059452 | 2055831 | 53 |
| chr4 | 4 | NA | − | 5509 | −1 | 54 |
| chr1 | 6 | NA | + | 128745 | 119073 | 55 |
| chr4 | 30 | NA | − | 47335 | 45352 | 56 |
| chr10 | 17 | inside | − | 112112 | 111645 | 57 |
| | | transcription | | | | |
| | | region | | | | |
| chr8 | 14 | NA | + | 241904 | 236381 | 58 |
| chr11 | 9 | inside | + | 82829 | 80232 | 59 |
| | | transcription | | | | |
| | | region | | | | |
| chr12 | 9 | inside | − | 831941 | 829464 | 60 |
| | | transcription | | | | |
| | | region | | | | |
| chr7 | 23 | inside | − | 1048731 | 1046953 | 61 |
| | | transcription | | | | |
| | | region | | | | |
| chr1 | 19 | inside | − | 193631 | 106881 | 62 |
| | | transcription | | | | |
| | | region | | | | |
| chr7 | 23 | inside | − | 1048731 | 1046953 | 63 |
| | | transcription | | | | |
| | | region | | | | |
| chr1 | 5 | inside | + | 280342 | 1772 | 64 |
| | | transcription | | | | |
| | | region | | | | |
| chr10 | 5 | NA | + | 300329 | 299781 | 65 |
| chr2 | 5 | NA | − | 102330 | 100705 | 66 |
| chr8 | 6 | NA | − | 65585 | −1 | 67 |
| chr6 | 5 | NA | − | 9344 | 6338 | 68 |
| chr4 | 74 | inside | + | 486699 | 483114 | 69 |
| | | transcription | | | | |
| | | region | | | | |
| chr11 | 9 | inside | + | 82829 | 80232 | 70 |
| | | transcription | | | | |
| | | region | | | | |
| chr8 | 14 | NA | − | 38691 | 29086 | 71 |
| chr14 | 11 | 3′ UTR | − | 95487 | 53778 | 72 |
| chr8 | 4 | NA | − | 20028 | 917 | 73 |
| chr12 | 18 | inside | | 283933 | 261207 | 74 |
| | | transcription | | | | |
| | | region | | | | |
| chr9 | 24 | inside | + | 314550 | 187324 | 75 |
| | | transcription | | | | |
| | | region | | | | |
| chr8 | 6 | 5' UTR | + | 65585 | −1 | 76 |
| chr9 | 8 | NA | + | 23177 | 10297 | 77 |
| chr12 | 12 | NA | + | 588014 | 231604 | 78 |
| chr16 | 14 | NA | + | 380589 | 377622 | 79 |
| chr3 | 10 | NA | − | 32890 | 21284 | 80 |
| chr6 | 13 | NA | + | 226842 | 225067 | 81 |
| chr19 | 8 | inside | − | 24555 | 24154 | 82 |
| | | transcription | | | | |
| | | region | | | | |
| chr10 | 27 | NA | − | 35083 | 34741 | 83 |
| chr2 | 29 | inside | − | 42643 | 42215 | 84 |
| | | transcription | | | | |
| | | region | | | | |
| chr8 | 6 | 5′ UTR | + | 65585 | −1 | 85 |
| chr4 | 4 | NA | − | 5509 | −1 | 86 |
| chr1 | 8 | inside | + | 77552 | 63148 | 87 |
| | | transcription | | | | |
| | | region | | | | |
| chr5 | 17 | NA | + | 176283 | 143845 | 88 |
| chr10 | 21 | inside | + | 122040 | 119961 | 89 |
| | | transcription | | | | |
| | | region | | | | |
| chr6 | 36 | inside | + | 341108 | 339787 | 90 |
| | | transcription | | | | |
| | | region | | | | |
| chr17 | 10 | NA | − | 446767 | 441180 | 91 |
| chr8 | 70 | inside | − | 2059452 | 2055831 | 92 |
| | | transcription | | | | |
| | | region | | | | |
| chr10 | 37 | inside | − | 415476 | 412253 | 93 |
| | | transcription | | | | |
| | | region | | | | |
| chr16 | 9 | inside | + | 1113012 | 1112017 | 94 |
| | | transcription | | | | |
| | | region | | | | |

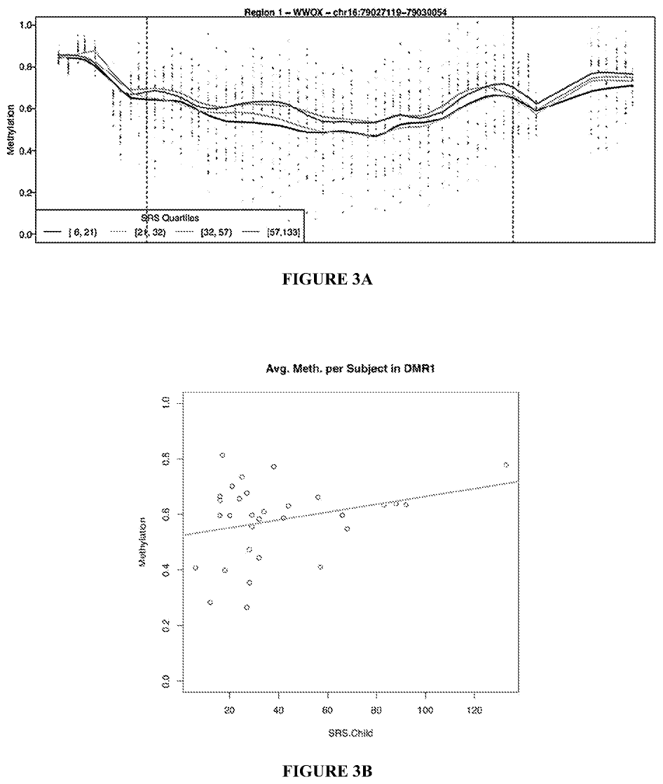

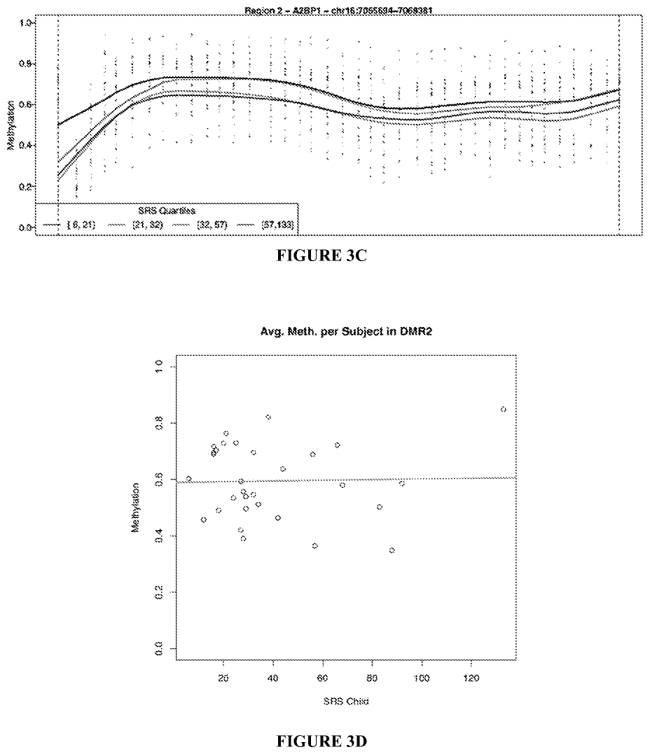

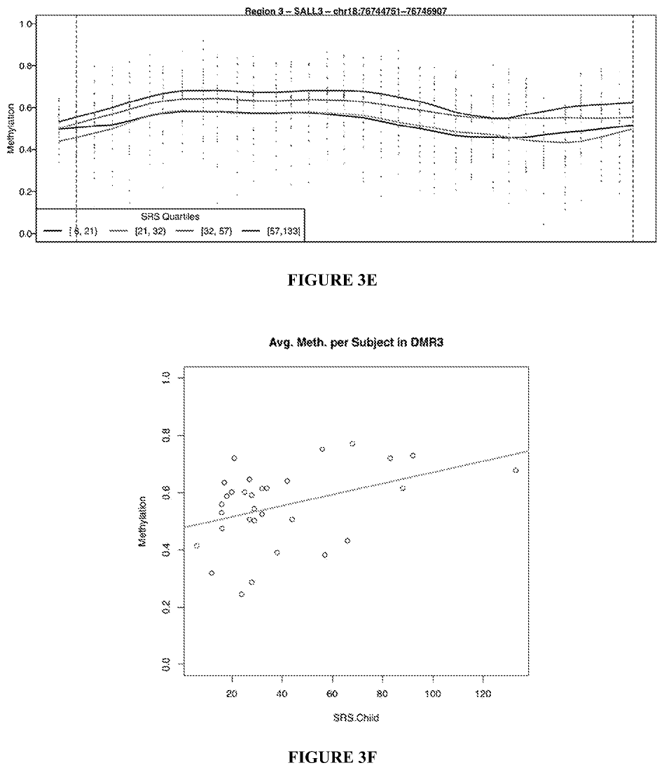

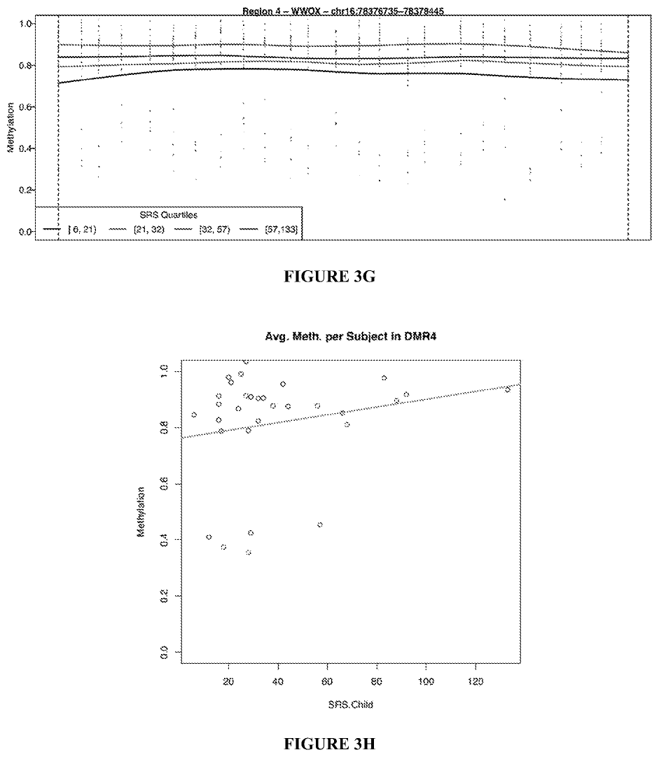

We highlight the top four regions where DNA methylation was significantly associated with SRS scores in children (p<0.05, FIG. 3), with the relationship between the average methylation level for each individual and SRS scores plotted in the inset for each panel. FIGS. 3A-B illustrate that increasing levels of DNA methylation are associated with higher SRS scores in children at the WW Doman Containing Oxidoreductase (WWOX) gene (p=0.00). Specifically, there was a 5.5% difference in average methylation between the highest (Q4) and lowest (Q1) SRS quartiles for this region. We see a similar positive linear association between DNA methylation and SRS scores for Spalt Like Transcription Factor 3 (SALL3) where there was an average of 10% methylation difference between SRS Q1 and Q4, (p-0.00, FIGS. 3E-F) as well as for a second region of WWOX where there was an average of 8% methylation difference across SRS Q1 and Q4 (p-0.00, FIGS. 3G-H illustrate the relationship between WWOX methylation and SRS score.). Interestingly, four distinct DMRs associated with WWOX, one of the SFARI autism genes, are within the top 20 significant DMRs associated with child SRS scores. FIGS. 3C-D represent the region of RNA Binding Fox-1 Homolog 2 (A2BP1/RBFOX1), another gene from the SFARI list, that was significantly associated with child SRS scores (p-0.00), which also overlaps with the SFARI Gene database. Here, we see that higher levels of methylation are associated with lower SRS scores, with an average methylation difference of 7% between SRS Q1 and Q4. Of the 94 significant DMRs, 68 (72.3%) have a positive relationship between paternal DNA methylation and child SRS scores.

### DNA Methylation and Paternal SRS Score

We next asked whether DMRs in sperm were associated with paternal SRS scores. Using the same bump-hunting method that was applied to the sperm methylation and child SRS data, we identified distinct set of 1928 DMRs in sperm that were associated with paternal SRS scores. Of those, 14 achieved genome-wide significance after permutation analyses (family-wise error rate (fwer) p<0.05, FIG. 2B). Table 8 lists the 14 DMRs that were significantly associated with paternal SRS scores (p<0.05). Genes in this list have shared functions associated with epigenetic regulation, embryonic development, cellular differentiation, and neuronal signaling.

TABLE 8
Dad DMRs with functions and ASD-associations (known or new)

| | | | | | Known or Novel |
|---|---|---|---|---|---|
| Genomic | | | Genic | | Association with |
| Location | FWER | Symbol | Location | Function | ASD |
| chr1: 246058026- | 8.00E−04 | SMYD3 | inside | Histone methyltransferase | Previously known |
| 246059550 | | | intron | | association |
| chr18: 76744751- | 0.002 | SALL3 | inside | Inhibits DNMT3A function at | Previously known |
| 76746907 | | | intron | CpG islands; roles in embryonic | association |
| | | | | development | |
| chr10: 114073582- | 0.0032 | GUCY2G | covers | Potentially involved in | novel association |
| 114075044 | | | exon(s) | mediating sperm-oocyte | |
| | | | | interactions | |
| chr20: 2216856- | 0.0066 | TGM3 | upstream | Transglutaminase enzyme | Previously known |
| 2218239 | | | | involved in protein corss-linking | association |
| | | | | in differentiated keratinocytes | |
| chr2: 905862- | 0.0091 | C2orf90 | overlaps 5′ | limited literature | novel association |
| 907360 | | | | | |
| chr16: 79027537- | 0.0112 | WWOX | inside | Oxidoreductase involved in | Previously known |
| 79029698 | | | intron | DNA damage repair | association |
| | | | | mechanisms and neuronal | (SFARI Category |
| | | | | signaling | 2) |
| chr17: 37756945- | 0.0176 | NEUROD2 | downstream | Transcriptional regulator | Previously known |
| 37758433 | | | | invovled in neuronal | association |
| | | | | differentiation | |
| chr10: 44173499- | 0.0178 | ZNF32 | upstream | Regulation of transcription by | novel association |
| 44175059 | | | | RNA polymerase II | |
| chrX: 63444648- | 0.0199 | ASB12 | overlaps 5′ | Involved in E3 ubiquitin ligase- | novel association |
| 63446044 | | | | mediated protein degradation | |
| chr5: 1973342- | 0.0222 | IRX4 | upstream | Mediates ventricular | Previously known |
| 1974948 | | | | differentiation during cardiac | association |
| | | | | development | |
| chr1: 5035511- | 0.028 | AJAP1 | downstream | Involved in synaptic functioning | Previously known |
| 5037033 | | | | | association |
| chr2: 59475581- | 0.0467 | FANCL | upstream | Ubiquitin ligase, involved in | Previously known |
| 59477094 | | | | neuropeptide signaling in the | association |
| | | | | brian | |
| chr11: 134034267- | 0.0473 | NCAPD3 | inside | Involved in cell cycle regulation | Previously known |
| 134035655 | | | intron | and cell division | association |
| chr2: 12880585- | 0.0474 | TRIB2 | inside exon | Largely unknown function; part | novel association |
| 12881771 | | | | of the Tribbles family of | |
| | | | | proteins | |

The top 14 significant DMRs in paternal sperm associated with SRS scores in fathers. The boundaries of the DMR are shown in the genomic location column; the fwer p value is displayed alongside the gene symbol. The genic location characterized where within the gene body the DMR is located. Gene functions were taken from the human protein atlas (proteinatlas.org) as well as gene cards (genecards.org). Associations with ASD were determined by literature describing associations of the gene with autism. SFARI category is defined by the Simons Foundation for Autsim Research Initiative (SFARI) based on their scoring algorithm. A score of 2 reflects a strong candidate. Genes that did not meet this criteria are termed “novel association”.

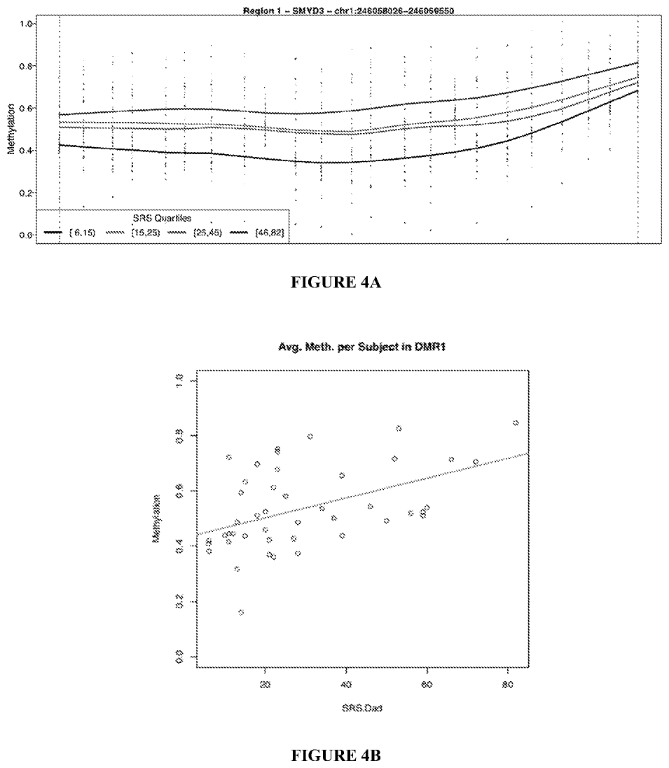

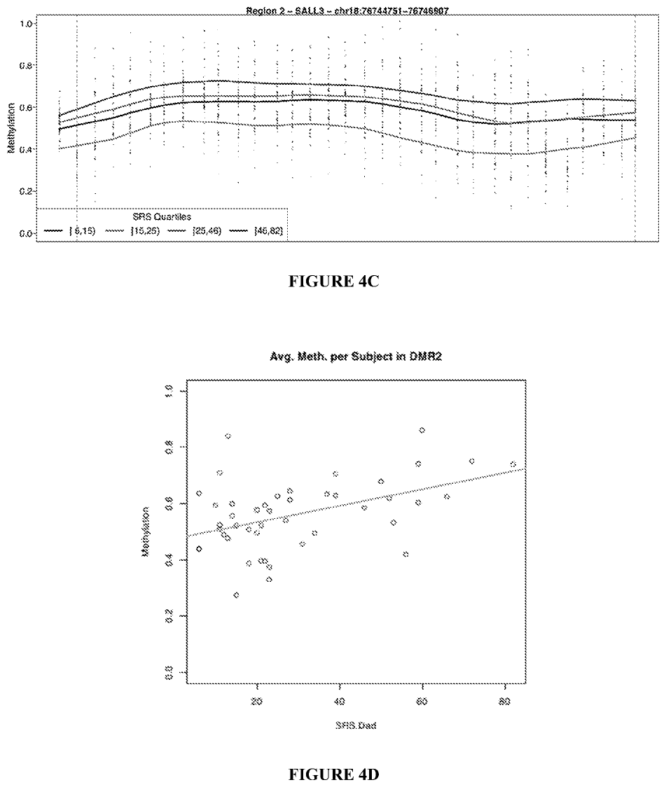

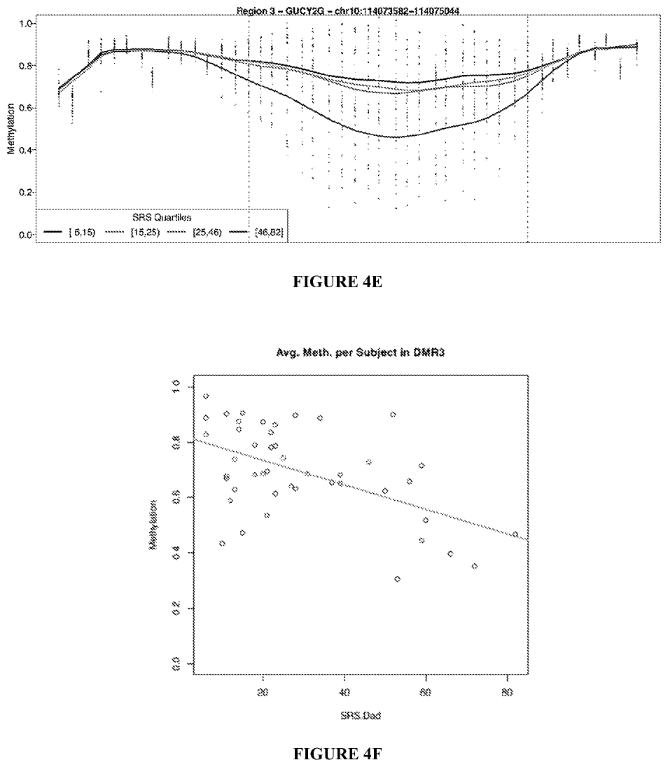

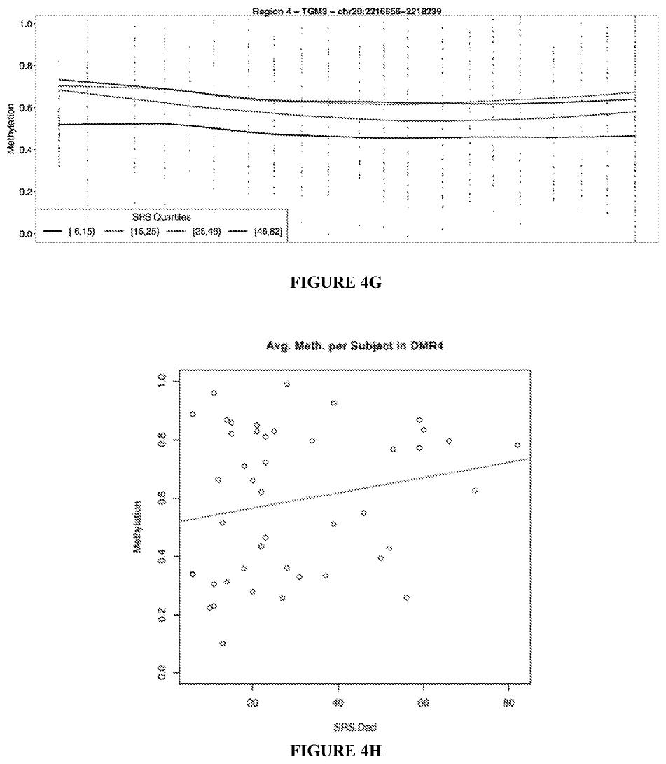

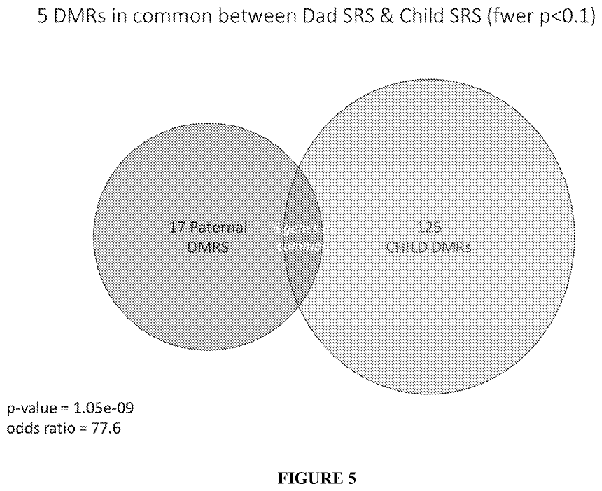

Presented here are the top four regions where DNA methylation significantly associates with paternal SRS scores (p<0.05, FIG. 4). For SET and MYND Domain Containing (SMYD3, p-0.0008, FIGS. 4A-B), SALL3 (p-0.002, FIGS. 4C-D), and Transglutaminase 4 (TGM3, p-0.0066, FIGS. 4G-H) there is a significant positive association between DNA methylation and paternal SRS scores. The effect sizes here are also of note, with an average methylation difference of 19.5% across the first and fourth quartiles of SRS scores for SMYD3; an average 8.3% methylation difference across SRS Q1 and Q4 for SALL3; and an average methylation difference of 16.5% at TGM3. For Guanylate Cyclase 2G (GUCY2G, p-0.0032, FIGS. 4E-F), however, there is a significant inverse relationship between sperm DNA methylation and paternal SRS scores. There is an average interindividual methylation difference of 19.8% across the first and fourth quartiles of SRS scores for GUCY2G. Overall, for the 14 significant DMRs, we observed that for the majority, 10 (71.4%), there was a significant positive association between DNA methylation and paternal SRS score.

### Comparison Across Datasets

### CHARM SRS Child and Dad

A subset of DMRs were similarly associated with child and paternal SRS scores. Here, we expanded our analysis threshold and took the lists of DMRs with fwer p<0.1 (n=23 genes for paternal SRS, and n-131 for child SRS) given that the fwer p-value is a conservative significance threshold. Across these two lists, we found six gene in common (FIG. 5, Fishers p=1.05×10-9, Odds Ratio (OR)-77.6), of which, five had overlapping DMRs. The genes associated with those five DMRs were WWOX, SALL3, Adherens Junctions Associated Protein 1 (AJAP1), TGM3, and Iroquois Homeobox 4 (IRX4).

Comparison with Independent Datasets

### CHARM SRS Child and AOSI

To determine if there was a consistent association between paternal sperm methylation and autism-related outcomes in children we compared DMRs that were significantly associated with child SRS scores at 36 months with DMRs that were significantly associated with AOSI scores at 12 months in the same children. There were 16 genes in common between 12-month AOSI and child SRS scores (fishers p=2.2×10-16, OR=44.5). Additionally, comparison of Gene Ontology biological process terms associated with the SRS-DMRs and AOSI-DMRs identified a number of overlapping terms including: “nervous system development”, “locomotion”, “generation of neurons”, “neurogenesis”, and “chemical synaptic transmission” (Table 9).

TABLE 9
DMRs associated with SRS scores.

| GOBPID | Pvalue | OddsRatio | ExpCount | Count | Size | Term |
|---|---|---|---|---|---|---|
| GO: 0007399 | 0.000415054 | 2.917841321 | 7.819347942 | 18 | 2310 | nervous |
| | | | | | | system |
| | | | | | | development |
| GO: 0009611 | 0.001166102 | 4.263097161 | 2.122394441 | 8 | 627 | response to |
| | | | | | | wounding |
| GO: 0001894 | 0.001440834 | 6.599505267 | 0.832709781 | 5 | 246 | tissue |
| | | | | | | homeostasis |
| GO: 0030168 | 0.001867476 | 8.312382856 | 0.524674862 | 4 | 155 | platelet |
| | | | | | | activation |
| GO: 0048666 | 0.003298105 | 3.093380615 | 3.689649029 | 10 | 1090 | neuron |
| | | | | | | development |
| GO: 0048699 | 0.003406458 | 2.788077361 | 4.999643684 | 12 | 1477 | generation |
| | | | | | | of neurons |
| GO: 0006928 | 0.00361687 | 2.503750426 | 7.14234812 | 15 | 2110 | movement of |
| | | | | | | cell or |
| | | | | | | subcellular |
| | | | | | | component |
| GO: 0060249 | 0.003911289 | 4.359343737 | 1.513094602 | 6 | 447 | anatomical |
| | | | | | | structure |
| | | | | | | homeostasis |
| GO: 0007596 | 0.004786563 | 4.946514423 | 1.10012471 | 5 | 325 | blood |
| | | | | | | coagulation |
| GO: 0007599 | 0.005038705 | 4.884259259 | 1.113664707 | 5 | 329 | hemostasis |
| GO: 0050817 | 0.00510319 | 4.868934911 | 1.117049706 | 5 | 330 | coagulation |
| GO: 0050878 | 0.005519006 | 4.047654505 | 1.624799572 | 6 | 480 | regulation |
| | | | | | | of body |
| | | | | | | fluid levels |
| GO: 0048468 | 0.005519207 | 2.44235527 | 6.729378229 | 14 | 1988 | cell |
| | | | | | | development |
| GO: 0022008 | 0.006135212 | 2.571126612 | 5.378763585 | 12 | 1589 | neurogenesis |
| GO: 0048731 | 0.006935978 | 2.027434126 | 15.88580082 | 25 | 4693 | system |
| | | | | | | development |
| GO: 0003008 | 0.00711255 | 2.363349203 | 6.925708177 | 14 | 2046 | system process |
| GO: 0007268 | 0.007133983 | 3.409063444 | 2.264564404 | 7 | 669 | chemical |
| | | | | | | synaptic |
| | | | | | | transmission |
| GO: 0098916 | 0.007133983 | 3.409063444 | 2.264564404 | 7 | 669 | anterograde |
| | | | | | | trans-synaptic |
| | | | | | | signaling |
| GO: 0040011 | 0.007443796 | 2.418457979 | 6.228398361 | 13 | 1840 | locomotion |
| GO: 0099537 | 0.00753699 | 3.371928251 | 2.288259398 | 7 | 676 | trans-synaptic |
| | | | | | | signaling |
| GO: 0042060 | 0.007567524 | 3.776540202 | 1.736504543 | 6 | 513 | wound healing |
| GO: 0050877 | 0.007817046 | 2.706807467 | 4.173703902 | 10 | 1233 | nervous |
| | | | | | | system process |
| GO: 0002009 | 0.008137603 | 3.716047973 | 1.763584536 | 6 | 521 | morphogenesis |
| | | | | | | of an |
| | | | | | | epithelium |
| GO: 0099536 | 0.008848148 | 3.265043478 | 2.359344379 | 7 | 697 | synaptic |
| | | | | | | signaling |
| GO: 0003018 | 0.009190502 | 5.22396779 | 0.822554784 | 4 | 243 | vascular |
| | | | | | | process in |
| | | | | | | circulatory |
| | | | | | | system |
| GO: 0016477 | 0.009550045 | 2.505799084 | 4.986103688 | 11 | 1473 | cell migration |

### CHARM SRS Child and Postmortem ASD Brain

Lastly, to ascertain functional relevance of DMRs in sperm that were associated with child SRS scores we sought to determine how many of these DMRs were similarly differentially methylated in postmortem brain tissues of individuals with autism compared to controls. DNA methylation data was measured on the 450K by Ladd-Acosta et al in three brain regions—the cerebellum, the prefrontal cortex, and the temporal cortex33. A subset of the significant child SRS-associated DMRs had at least one probe from the 450K data that overlapped the CHARM region. Specifically, we observed six child SRS-DMRs that were significantly differentially methylated between autism cases and controls in the cerebellum annotated to the following genes: SALL3, PFKP, CUBN, FRMD1, SYT1, and PITRM1; a second set of six child SRS-DMRs that were significantly differentially methylated in the prefrontal cortex: PFKP, GIMAP7, FRMD1, A2BP1, C1ORF140, and APOB; and 10 child SRS-DMRs that were significantly differentially methylated in the temporal cortex: IRX2, SOX9, FRMD1, IRX3, CNTN4, C1ORF150, CX3CR1, BTBD7, SMOC2, and LRRC16A.

### Example 3

### Discussion

The main objective of this work was to investigate potential paternal contributions to autistic traits in children. SRS scores, a measure of autistic traits, were not significantly correlated between fathers and children. We identified methylation changes in paternal sperm that were significantly associated with child SRS scores. Many of those DMRs were annotated to genes implicated in ASD and neurodevelopment. Some of these child SRS-associated DMRs were similarly associated with paternal SRS scores, though many were distinctly associated with either child SRS or paternal SRS scores. Comparison to earlier work revealed that several DMRs associated with child SRS scores at 36-months were similarly associated with AOSI scores in the same children at 12-months. We further demonstrated that DMRs associated with child SRS scores contained CpG sites that were independently found to be differentially methylated in multiple brain regions from postmortem brain tissues of individuals with and without autism. Together, this data provides compelling support for the role of the association between paternal epigenetic information and ASD-related traits in children.

There is a need to consider paternal contributions to autism more strongly. One prevailing hypothesis for paternal contributions to offspring ASD susceptibility is advanced paternal age. This work has consistently demonstrated that the risk of ASD diagnoses increase with advanced paternal aging, due in part to increased rates of spontaneous de novo mutations, decreased efficacy of DNA proofreading and repair enzymes, and increased rates of DNA fragmentation. Other studies have shown that in vitro fertilization by intracytoplasmic sperm injection (ICS) increases the relative risk of ASD by nearly 5%, highlighting another possible paternal contribution. Though the mechanism responsible for the increased risk of ASD following ICSI remains elusive, it is plausible to hypothesize that it could be due to inherent genetic abnormalities impeding fertilization, or epigenetic changes resulting from artificial conditions. Epigenetic changes in sperm are especially important to consider in autism etiology. Such changes often represent an interplay of genetics and the environment, and studies are increasingly demonstrating that the sperm methylome responds to environmental exposures, particularly at genes important for neurodevelopment. Most recently, a DNA methylation signature in sperm was developed for use as a biomarker to identify potential for paternal epigenetic contributions to ASD. To our knowledge, the present study was the first to examine how changes to the sperm methylome are associated with SRS scores in offspring with a higher likelihood of developing ASD compared to the general population. This work, building on the expanding body of literature described above, reinforces the need to further investigate paternal epigenetic contributions to autism, and to include these paternal assessments in epidemiologic studies of ASD risk.

We focused on the 36-month child SRS developmental assessment as a measure of early ASD-related phenotypes in the EARLI cohort. Of the children included in this analysis, 9 of them had received an ASD diagnosis at 36 months based on the Baby Siblings Research Consortium diagnostic criteria (41), and their 36-month SRS scores ranged from 16-133. Others have reported evidence for heritability, specifically paternal heritability, of SRS scores as a representation of the heritability of autistic phenotypes. In this study, we did not observe this pattern of heritability of SRS scores themselves, possibly due to a small sample size, or to the autism enriched familial design of the EARLI cohort.

Among the DMRs significantly associated with child SRS scores, we found that many either had previously known roles in autism or neurodevelopment and brain functioning more broadly. One gene of particular interest was WWOX. WWOX is among the largest genes in the human genome and is located at a common fragile site, FRA16D, a hotspot for germline variants associated with neuropathogenicity. Copy number variations across the WWOX locus were identified in children with autism from families with multiple individuals with ASD diagnoses, and additional point mutations and deletions within the gene have been associated with autism and intellectual disability more broadly. Remarkably there were five distinct DMRs within this gene that were significantly associated with child SRS scores, one of which was similarly associated with paternal SRS scores. According to the SFARI database, this gene is classified as a strong autism candidate gene. That we see multiple regions of this gene significantly associated with SRS scores in both children and fathers suggests that epigenetic regulation of this gene, either in concert with, or independent of, genetic variation, is also associated with autistic traits.

While we did not observe a correlation between AOSI and SRS scores themselves, we did find that a significant number of DMRs in sperm were associated with both of these autistic traits. It is not surprising that there was not a correlation between AOSI and SRS given that they are measuring different phenotypes at different stages of life. The AOSI is a behavioral assessment while the SRS is an assessment of social cognition and communication. Similarly, given that they are measuring different phenotypes, it is not unexpected that the DMRs most significantly associated with each trait are distinct from one another. However, that we do observe commonalities in sperm DMRs associated with both traits suggests that methylation patterns in paternal sperm that are associated with these offspring ASD-related phenotypes might have shared underlying biological pathways that are important for neurodevelopment. This was supported by the finding that there were multiple Biological Process GO terms associated with both SRS-DMRs and AOSI-DMRs that are involved in neurodevelopment. This reinforces the notion that DMRs in sperm that are associated with these different phenotypes share common biological pathways involved in neurodevelopment. This is an important area for future investigation.

Our comparison with postmortem tissues highlighted the functional relevance of the associations between DMRs in sperm and child SRS scores, as multiple DMRs were similarly differentially methylated between individuals with and without autism in the cerebellum, the temporal cortex, and the prefrontal cortex. This is compelling given that functional and anatomical changes in these regions are reported in individuals with ASD. It is of intense interest to uncover whether methylation changes in sperm can resist epigenetic reprogramming and contribute epigenetic information to the developing embryo. This understanding would help explain how the methylation changes in sperm that associate with autistic traits might impact neurodevelopment by altering the epigenome of offspring brains. Parts of the genome do escape epigenetic reprograming, rendering them strong candidates for regions where intergenerational heritability may occur.

It is also possible that there is an interplay of genetic and epigenetic contributions to ASD, where genetic contributions are mediated by gametic epigenetic changes. Methylation quantitative trait loci (meQTLs) represent regions of the genome where genetic variations can influence patterns of DNA methylation at specific loci. Studies have identified single nucleotide polymorphisms (SNPs) associated with ASD that regulate site-specific DNA methylation changes across cord blood, peripheral blood, and fetal brain tissues. Whether meQTLs exist in sperm to regulate DNA methylation in a potentially heritable manner, is an important area for future investigation. Recent work in postmortem brain tissues demonstrated that methylation differences across regions in normal brains are enriched for psychiatric genomics consortium-identified SNPs that have been associated with neuropsychiatric trait heritability. In this study, DMRs identified in neuronal cells were significantly associated with the heritability of neuropsychiatric conditions including schizophrenia, ADHD, and neuroticism. These findings demonstrate that genetic signals associated with neuropsychiatric traits are mediated through epigenetic modifications. This suggests that meQTLs, possibly influenced by or independent of, the environment, may account for neuropsychiatric germline transmission. This is consistent with findings from this study if we assume that DNA methylation is an epigenetic marker of risk. Further, that there were DMRs associated only with paternal SRS scores warrants further investigation into the potential interaction between genetic and epigenetics in autism heritability. Lastly, it is important to consider that while this work focused on DNA methylation alone, the epigenetic landscape is marked by multiple modifications working in concert to regulate DNA accessibility, chromatin architecture, and gene expression. Thus, it is possible a combination of epigenetic factors might be working together, either alongside or independent of genetics, to contribute to ASD risk in the next generation.

One potential limitation to this work is that DNA methylation analyses were performed on DNA that was extracted from semen, not purified mature motile sperm. Thus, it is possible that our findings may have been diluted by cellular heterogeneity. Nevertheless, we detected meaningful associations between sperm methylation and paternal and child SRS scores at genes relevant for neurodevelopment. Additionally, our sample sizes were relatively small so we may have been underpowered to detect all meaningful changes and associations in our data. This is in part due to the general lack of inclusion of sperm sample collection in autism epidemiology cohorts, and this paper will hopefully encourage other groups to collect these critical samples. However, the nature of the EARLI cohort renders our findings internally valid as there is a precedent for cohorts designed with increased familial likelihood in the epidemiologic literature for disorders with familial clustering like ASD. Lastly, the CHARM design covers regions of the genome that have at least moderate CpG density and does not account for individual CpG level methylation. Thus, while millions of CpG sites are included in the DMRs that CHARM measures, it is possible that we did not gather methylation information at regions that are less densely populated with CpG sites but still play important regulatory roles. Despite this, the CHARM array is advantageous for identifying regional methylation changes across the genome. Moreover, the sample size limitation may be outweighed by our focus on a quantitative trait, SRS, which allowed us to observe genome-wide significant DMRs (with rigorous correction for multiple testing), that may not be easily achieved in case-control designs.

The limitations to this work are balanced by the strength of our findings. This was the first study to demonstrate that paternal sperm DNA methylation is associated with SRS scores in 36-month-old children, particularly at genes with roles in neurodevelopment and known involvement in autism. As such, our study might contribute insight into the etiology of ASD-associated social communication deficits rather than the ASD diagnosis itself. These findings were present in the younger siblings of children who had previously been diagnosed with ASD, thus increasing their likelihood of also being diagnosed. Many of the DMRs that were associated with both paternal and child SRS scores were involved in neurodevelopment and early development, and there were meaningful commonalities between child SRS-associated DMRs and CpG sites that were associated with ASD in postmortem brain samples. In an area that has been overlooked for too long, this work contributes the important finding that epigenetic changes in sperm at genes important for neurodevelopment are associated with autistic traits in fathers and their children. This underscores the urgent need to evaluate paternal epigenetic contributions to autism, an area that has been overlooked for too long.

Although the invention has been described with reference to the above examples, it will be understood that modifications and variations are encompassed within the spirit and scope of the invention. Accordingly, the invention is limited only by the following claims.

### LIST OF REFERENCES

1. Hertz-Picciotto I, Schmidt RJ, Krakowiak P. Understanding environmental contributions to autism: Causal concepts and the state of science. Autism Res. 2018; 11 (4): 554-86.
2. Havdahl A, Niarchou M, Starnawska A, Uddin M, van der Merwe C, Warrier V. Genetic contributions to autism spectrum disorder. Psychol Med. 2021; 51 (13): 2260-73.
3. Lyall K, Constantino JN, Weisskopf MG, Roberts AL, Ascherio A, Santangelo SL. Parental social responsiveness and risk of autism spectrum disorder in offspring. JAMA Psychiatry. 2014; 71 (8): 936-42.
4. Bhandari R, Paliwal JK, Kuhad A. Neuropsychopathology of Autism Spectrum Disorder: Complex Interplay of Genetic, Epigenetic, and Environmental Factors. Adv Neurobiol. 2020; 24:97-141.
5. Donkin I, Barres R. Sperm epigenetics and influence of environmental factors. Mol Metab. 2018; 14:1-11.
6. Waye MMY, Cheng HY. Genetics and epigenetics of autism: A Review. Psychiatry Clin Neurosci. 2018; 72 (4): 228-44.
7. Greenberg MVC, Bourc'his D. The diverse roles of DNA methylation in mammalian development and disease. Nat Rev Mol Cell Biol. 2019; 20 (10): 590-607.
8. Feinberg JI, Bakulski KM, Jaffe AE, Tryggvadottir R, Brown SC, Goldman LR, et al. Paternal sperm DNA methylation associated with early signs of autism risk in an autism-enriched cohort. Int J Epidemiol. 2015; 44 (4): 1199-210.
9. Garrido N, Cruz F, Egea RR, Simon C, Sadler-Riggleman I, Beck D, et al. Sperm DNA methylation epimutation biomarker for paternal offspring autism susceptibility. Clin Epigenetics. 2021; 13 (1): 6.
10. McSwiggin H M, O'Doherty AM. Epigenetic reprogramming during spermatogenesis and male factor infertility. Reproduction. 2018; 156 (2): R9-R21.
11. Stewart KR, Veselovska L, Kelsey G. Establishment and functions of DNA methylation in the germline. Epigenomics. 2016; 8 (10): 1399-413.
12. Trasler JM. Epigenetics in spermatogenesis. Mol Cell Endocrinol. 2009; 306 (1-2): 33-6.
13. Uysal F, Akkoyunlu G, Ozturk S. DNA methyltransferases exhibit dynamic expression during spermatogenesis. Reprod Biomed Online. 2016; 33 (6): 690-702.
14. Wu H, Hauser R, Krawetz SA, Pilsner JR. Environmental Susceptibility of the Sperm Epigenome During Windows of Male Germ Cell Development. Curr Environ Health Rep. 2015; 2 (4): 356-66.
15. Zhang W, Yang J, Lv Y, Li S, Qiang M. Paternal benzo[a]pyrene exposure alters the sperm DNA methylation levels of imprinting genes in F0 generation mice and their unexposed F1-2 male offspring. Chemosphere. 2019; 228:586-94.
16. Murphy SK, Itchon-Ramos N, Visco Z, Huang Z, Grenier C, Schrott R, et al. Cannabinoid exposure and altered DNA methylation in rat and human sperm. Epigenetics. 2018; 13 (12): 1208-21.
17. Jenkins TG, James ER, Alonso DF, Hoidal JR, Murphy PJ, Hotaling JM, et al. Cigarette smoking significantly alters sperm DNA methylation patterns. Andrology. 2017; 5 (6): 1089-99.
18. Morkve Knudsen GT, Rezwan FI, Johannessen A, Skulstad SM, Bertelsen RJ, Real FG, et al. Epigenome-wide association of father's smoking with offspring DNA methylation: a hypothesis-generating study. Environ Epigenet. 2019; 5 (4): dvz023.
19. Consales C, Toft G, Leter G, Bonde J P, Uccelli R, Pacchierotti F, et al. Exposure to persistent organic pollutants and sperm DNA methylation changes in Arctic and European populations. Environ Mol Mutagen. 2016; 57 (3): 200-9.
20. Donkin I, Versteyhe S, Ingerslev LR, Qian K, Mechta M, Nordkap L, et al. Obesity and Bariatric Surgery Drive Epigenetic Variation of Spermatozoa in Humans. Cell Metab. 2016; 23 (2): 369-78.
21. Virkud YV, Todd RD, Abbacchi AM, Zhang Y, Constantino JN. Familial aggregation of quantitative autistic traits in multiplex versus simplex autism. Am J Med Genet B Neuropsychiatr Genet. 2009; 150B (3): 328-34.
22. Sandin S, Lichtenstein P, Kuja-Halkola R, Larsson H, Hultman CM, Reichenberg A. The familial risk of autism. JAMA. 2014; 311 (17): 1770-7.
23. Maenner MJSK, Bakian AV et al. Prevalence and Characteristics of Autism Spectrum Disorder Among Children Aged 8 Years-Autism and Developmental Disabilities Monitoring Network, 11 Sites, United States. MMWR Surveill Summ 2021. 2018; 70:1-16.
24. Constantino JN, Davis SA, Todd RD, Schindler MK, Gross MM, Brophy SL, et al. Validation of a brief quantitative measure of autistic traits: comparison of the social responsiveness scale with the autism diagnostic interview-revised. J Autism Dev Disord. 2003; 33 (4): 427-33.
25. Newschaffer CJ, Croen LA, Fallin MD, Hertz-Picciotto I, Nguyen DV, Lee NL, et al. Infant siblings and the investigation of autism risk factors. Journal of neurodevelopmental disorders. 2012; 4 (1): 7.
26. Wigham S, McConachie H, Tandos J, Le Couteur AS, Gateshead Millennium Study core t. The reliability and validity of the Social Responsiveness Scale in a UK general child population. Res Dev Disabil. 2012; 33 (3): 944-50.
27.Chan W, Smith LE, Hong J, Greenberg JS, Mailick MR. Validating the social responsiveness scale for adults with autism. Autism Res. 2017; 10 (10): 1663-71.
28. Ladd-Acosta C, Aryee MJ, Ordway JM, Feinberg AP. Comprehensive high-throughput arrays for relative methylation (CHARM). Curr Protoc Hum Genet. 2010; Chapter 20: Unit 20 1 1-19.
29.Chang CC, Chow CC, Tellier LC, Vattikuti S, Purcell SM, Lee JJ. Second-generation PLINK: rising to the challenge of larger and richer datasets. Gigascience. 2015; 4:7.
30.Patterson N, Price AL, Reich D. Population structure and eigenanalysis. PLOS Genet. 2006; 2 (12): e190.
31. Price AL, Patterson NJ, Plenge RM, Weinblatt ME, Shadick NA, Reich D. Principal components analysis corrects for stratification in genome-wide association studies. Nat Genet. 2006; 38 (8): 904-9.
32. Aryee MJ, WuZ, Ladd-Acosta C, Herb B, Feinberg AP, Yegnasubramanian S, et al. Accurate genome-scale percentage DNA methylation estimates from microarray data. Biostatistics. 2011; 12 (2): 197-210.
33. Leek JT, Storey JD. Capturing heterogeneity in gene expression studies by surrogate variable analysis. PLOS Genet. 2007; 3 (9): 1724-35.
34. Buja A, Eyuboglu N. Remarks on Parallel Analysis. Multivariate Behav Res. 1992; 27 (4): 509-40.
35. Jaffe AE, Murakami P, Lee H, Leek JT, Fallin MD, Feinberg AP, et al. Bump hunting to identify differentially methylated regions in epigenetic epidemiology studies. Int J Epidemiol. 2012; 41 (1): 200-9.
36. Ladd-Acosta C, Hansen KD, Briem E, Fallin MD, Kaufmann WE, Feinberg AP. Common DNA methylation alterations in multiple brain regions in autism. Mol Psychiatry. 2014; 19 (8): 862-71.
37. Mao Y, Van Auken K, Li D, Arighi CN, McQuilton P, Hayman GT, et al. Overview of the gene ontology task at BioCreative IV. Database (Oxford). 2014; 2014.
38. Falcon S, Gentleman R. Using GOstats to test gene lists for GO term association. Bioinformatics. 2007; 23 (2): 257-8.
39. Yao P, Lin P, Gokoolparsadh A, Assareh A, Thang MW, Voineagu I. Coexpression networks identify brain region-specific enhancer RNAs in the human brain. Nat Neurosci. 2015; 18 (8): 1168-74.
40. Wilkinson B, Grepo N, Thompson BL, Kim J, Wang K, Evgrafov OV, et al. The autism-associated gene chromodomain helicase DNA-binding protein 8 (CHD8) regulates non-coding RNAs and autism-related genes. Transl Psychiatry. 2015; 5: e568.
41. Ozonoff S, Young GS, Landa RJ, Brian J, Bryson S, Charman T, et al. Diagnostic stability in young children at risk for autism spectrum disorder: a baby siblings research consortium study. J Child Psychol Psychiatry. 2015; 56 (9): 988-98.
42. Hussain T, Liu B, Shrock MS, Williams T, Aldaz CM. WWOX, the FRA16D gene: A target of and a contributor to genomic instability. Genes Chromosomes Cancer. 2019; 58 (5): 324-38.
43. Banne E, Abudiab B, Abu-Swai S, Repudi SR, Steinberg DJ, Shatleh D, et al. Neurological Disorders Associated with WWOX Germline Mutations-A Comprehensive Overview. Cells. 2021; 10 (4).
44. Aldaz CM, Hussain T. WWOX Loss of Function in Neurodevelopmental and Neurodegenerative Disorders. Int J Mol Sci. 2020; 21 (23).
45. Eyler LT, Pierce K, Courchesne E. A failure of left temporal cortex to specialize for language is an early emerging and fundamental property of autism. Brain. 2012; 135 (Pt 3): 949-60.
46. Donovan AP, Basson MA. The neuroanatomy of autism—a developmental perspective. J Anat. 2017; 230 (1): 4-15.
47. Courchesne E, Mouton PR, Calhoun ME, Semendeferi K, Ahrens-Barbeau C, Hallet MJ, et al. Neuron number and size in prefrontal cortex of children with autism. JAMA. 2011; 306 (18): 2001-10.
48. Gkountela S, Zhang KX, Shafiq TA, Liao WW, Hargan-Calvopina J, Chen PY, et al. DNA Demethylation Dynamics in the Human Prenatal Germline. Cell. 2015; 161 (6): 1425-36.
49. Tang WW, Dietmann S, Irie N, Leitch HG, Floros VI, Bradshaw CR, et al. A Unique Gene Regulatory Network Resets the Human Germline Epigenome for Development. Cell. 2015; 161 (6): 1453-67.
50. Kobayashi H, Sakurai T, Imai M, Takahashi N, Fukuda A, Yayoi O, et al. Contribution of intragenic DNA methylation in mouse gametic DNA methylomes to establish oocyte-specific heritable marks. PLOS Genet. 2012; 8 (1): e1002440.
51. Smith ZD, Chan MM, Mikkelsen TS, Gu H, Gnirke A, Regev A, et al. A unique regulatory phase of DNA methylation in the early mammalian embryo. Nature. 2012; 484 (7394): 339-44.
52. Keyhan S, Burke E, Schrott R, Huang Z, Grenier C, Price T, et al. Male obesity impacts DNA methylation reprogramming in sperm. Clin Epigenetics. 2021; 13 (1): 17.
53. Schrott R, Murphy S K, Modliszewski J L, King DE, Hill B, Itchon-Ramos N, et al. Refraining from use diminishes cannabis-associated epigenetic changes in human sperm. Environ Epigenet. 2021; 7 (1): dvab009.
54. Greeson KW, Fowler KL, Estave PM, Kate Thompson S, Wagner C, Clayton Edenfield R, et al. Detrimental effects of flame retardant, PBB153, exposure on sperm and future generations. Sci Rep. 2020; 10 (1): 8567.
55. Maggio A G, Shu HT, Laufer BI, Bi C, Lai Y, LaSalle JM, et al. Elevated exposures to persistent endocrine disrupting compounds impact the sperm methylome in regions associated with autism spectrum disorder. Front Genet. 2022; 13:929471.
56.Andrews SV, Ellis SE, Bakulski KM, Sheppard B, Croen L A, Hertz-Picciotto I, et al. Cross-tissue integration of genetic and epigenetic data offers insight into autism spectrum disorder. Nat Commun. 2017; 8 (1): 1011.
57. Rizzardi LF, Hickey P F, Rodriguez DiBlasi V, Tryggvadottir R, Callahan CM, Idrizi A, et al. Neuronal brain-region-specific DNA methylation and chromatin accessibility are associated with neuropsychiatric trait heritability. Nat Neurosci. 2019; 22 (2): 307-16.

1. A method of determining a risk of having an offspring with autism spectrum disorder (ASD) comprising:
a) measuring DNA methylation status at differentially methylated regions (DMRs) in DNA from a semen sample from a paternal subject; and
b) determining a risk score based on DMRs methylation status,
thereby determining a risk of having an offspring with ASD.

2. The method of claim 1, wherein a difference in the DNA methylation status at one or more DMRs as compared to a control DNA methylation status is indicative of a higher likelihood of having an offspring with ASD than a likelihood of having an offspring with ASD as measured in the control DNA.

3. The method of claim 2, wherein the control DNA methylation status is a DNA methylation status at the one or more DMRs measured in a subject that is not at risk of having an offspring with ASD.

4. The method of claim 1, wherein the subject is a prospective parent.

5. The method of claim 1, wherein the subject has a risk factor for having an offspring with ASD.

6. The method of claim 1, wherein determining a risk of having an offspring with ASD comprises predicting a risk of having an offspring with features of autism as measured by a social responsiveness scale (SRS) score.

7. A method of diagnosing autism spectrum disorder (ASD) in a subject comprising:
a) measuring a DNA methylation status at one or more differentially methylated regions (DMRs) in a DNA sample from the subject; and
b) determining a risk score based on DMRs methylation status, thereby diagnosing ASD in the subject.

8. The method of claim 7, wherein a difference in the DNA methylation status at the one or more DMRs as compared to a control DNA methylation status is indicative of a higher likelihood of having ASD than a likelihood of having ASD as measured in the control DNA.

9. The method of claim 1, wherein the DMRs are in genes selected from group of genes set forth in Table 6, Table 7 or Table 8.

10. The method of claim 1, wherein the DMRs comprise at least 1, at least 2, at least 3, at least 4, at least 5, at least 6, at least 7, at least 8, at least 9, at least 10, at least 11, at least 12, at least 13, or 14 genes from Table 6.

11. The method of claim 1, wherein the DMRs comprise at least 1, at least 2, at least 3, at least 4, at least 5, at least 6, at least 7, at least 8, at least 9, at least 10, at least 11, at least 12, at least 13, or 14 genes from Table 8.

12. The method of claim 1, wherein the DMRs comprise 3 to 15 DMRs.

13. The method of claim 9, wherein the genes are ASD-associated genes.

14. The method of claim 1, wherein a difference in the DNA methylation status comprises hypomethylation, hypermethylation or a combination thereof.

15. (canceled)

16. The method of claim 1, wherein measuring DNA methylation status is by methylation specific PCR, bisulfite sequencing, capture bisulfite sequencing, whole genome bisulfite sequencing, pyrosequencing, single-strand conformation polymorphism (SSCP) analysis, restriction analysis, microarray technology, including bead microarray technology, or proteomics.

17. The method of claim 1, wherein the DNA methylation status at the one or more DMRs is associated with an SRS score in the subject.

18. The method of claim 1, wherein the DNA methylation status at the one or more DMRs is associated with an SRS score in the offspring.

19. A method of determining an association between exposure to an environmental factor in a subject and an increased risk of having an offspring with autism spectrum disorder (ASD) comprising:
a) measuring a first DNA methylation status at differentially methylated regions (DMRs) in DNA from a first semen sample from the subject prior to exposure to the environmental factor;
b) measuring a second DNA methylation status at DMRs in DNA from a second semen sample from the subject after to exposure to the environmental factor; and
c) comparing the first and the second methylation status at the DMRs;
thereby determining an association between exposure to an environmental factor and an increased risk of having an offspring with ASD.

20. The method of claim 19, wherein a change in the methylation status at DMRs between the first DNA methylation status and the second DNA methylation status is indicative of an association between the environmental factor and an increased risk of having an offspring with autism ASD.

21. The method of claim 1, wherein the DNA is from sperm in the semen sample.

22-23. (canceled)
